SEQUENCE LISTING (9 sequences)

SEQ ID NO: 1 - protein, 17 aa, Homo sapien
DCDLIYYDYEEDYYFDY

SEQ ID NO: 2 - protein, 16 aa, Homo sapien
DQFHWKNSNQIKILGN

SEQ ID NO: 3 - protein, 16 aa, Homo sapien
DQGSFLTKGPSKLNDR

SEQ ID NO: 4 - protein, 15 aa, Homo sapien
QFHWKNSNQIKILGN

SEQ ID NO: 5 - protein, 15 aa, Homo sapien
NSNQIKILGNQGSFL

SEQ ID NO: 6 - protein, 15 aa, Homo sapien
KILGNQGSFLTKGPS

SEQ ID NO: 7 - protein, 15 aa, Homo sapien
QGSFLTKGPSKLNDR

SEQ ID NO: 8 - protein, 15 aa, Homo sapien
TKGPSKLNDRADSRR

SEQ ID NO: 9 - protein, 15 aa, Homo sapien
KLNDRADSRRSLWDQ

(19) United States
(12) Patent Application Publication
(10) Pub. No.: US 2009/0092631 A1
(43) Pub. Date: Apr. 9, 2009

(54) GLYCOSYLATED SPECIFICITY EXCHANGERS THAT INDUCE AN ANTIBODY DEPENDENT CELLULAR CYTOTOXICITY (ADCC) RESPONSE

(75) Inventors: Matti Sallberg, Stockholm (SE); Anders Vahlne, Bastad (SE); Maria Perdomo, Marynummi (FI)

Correspondence Address: KNOBBE MARTENS OLSON & BEAR LLP, 2040 MAIN STREET, FOURTEENTH FLOOR, IRVINE, CA, 92614, US

(73) Assignee: TRIPEP AB, Huddinge (SE)

(21) Appl. No.: 12/055,187

(22) Filed: Mar. 25, 2008

Related U.S. Application Data
(60) Provisional application No. 60/920,263, filed on Mar. 26, 2007.

(51) Int. Cl. A61K 39/385; A61P 37/04

(52) U.S. Cl. 424/193.1

(57) ABSTRACT

The present invention is directed to ligand/receptor and antigen/antibody specificity exchangers comprising a saccharide or glycoconjugate. Methods of making these specificity exchangers and methods of using said specificity exchangers to treat or prevent human disease are described herein.

### CROSS-REFERENCE TO RELATED APPLICATIONS

This application claims the benefit of priority of U.S. Provisional Patent Application No. 60/920,263, filed Mar. 26, 2007.

### REFERENCE TO SEQUENCE LISTING

The present application is being filed along with a Sequence Listing in electronic format. The Sequence Listing is provided as a file entitled TRIPEP_{—}100A.TXT, created Mar. 19, 2008, which is 2.08 KB in size. The information in the electronic format of the Sequence Listing is incorporated herein by reference in its entirety.

### FIELD OF THE INVENTION

Aspects of the present invention relate to compositions and methods for preventing and treating human disease, including cancer, and those resulting from pathogens such as bacteria, yeast, parasites, fungus, viruses, and the like. More specifically, embodiments described herein concern the manufacture and use of specificity exchangers or adapters, which are glycopeptides that redirect natural antibodies that are present in a subject to a pathogen and induce an ADCC response.

### BACKGROUND OF THE INVENTION

The “specificity exchangers” or “adapters” are generally composed of two domains, a specificity domain and an antigenic domain. There are two general types of specificity exchangers differentiated by the nature of their specificity domains. (See e.g., U.S. Pat. No. 6,933,366, hereby expressly incorporated by reference in its entirety). The first type of specificity exchanger is an antigen/antibody specificity exchanger. Several different types of antigen/antibody specificity exchangers can be made. (See e.g., U.S. Pat. Nos. 5,869,232; 6,040,137; 6,245,895; 6,417,324; 6,469,143; and U.S. application Ser. Nos. 09/839,447 and 09/839,666; and International App. Nos. PCT/SE95/00468 and PCT/IB01/00844, all of which are hereby expressly incorporated by reference in their entireties).

Antigen/antibody specificity exchangers comprise an amino acid sequence of an antibody that specifically binds to an antigen (i.e., the specificity domain) joined to an amino acid sequence to which an antibody binds (i.e., the antigenic domain). Some specificity domains of antigen/antibody specificity exchangers comprise an amino acid sequence of a complementarity determining region (CDR), are at least 5 and less than 35 amino acids in length, are specific for HIV-1 antigens, or are specific for hepatitis viral antigens. Some antigenic domains of antigen/antibody specificity exchangers comprise a peptide having an antibody-binding region of viral, bacterial, or fungal origin, are at least 5 and less than 35 amino acids in length, or contain peptides (e.g., peptides comprising epitopes) that are obtained from polio virus, measles virus, hepatitis B virus, hepatitis C virus, or HIV-1.

A second type of specificity exchanger, the ligand/receptor specificity exchanger, is also composed of a specificity domain and an antigenic domain, however, the specificity domain of the ligand/receptor specificity exchanger comprises a ligand for a receptor that is present on a pathogen, as opposed to a sequence of an antibody that binds to an antigen. That is, a ligand/receptor specificity exchanger differs from an antibody/antigen specificity exchanger in that the ligand/receptor specificity exchanger does not contain a sequence of an antibody that binds an antigen but, instead, adheres to the pathogen vis a vis ligand interaction with a receptor that is present on the pathogen. Several different types of ligand/receptor specificity exchangers can be made. (See e.g., U.S. Pat. Nos. 6,660,842 and 6,933,366, all of which are hereby expressly incorporated by reference in their entireties).

Some specificity domains of ligand/receptor specificity exchangers comprise an amino acid sequence that is a ligand for a bacterial adhesion receptor (e.g., extracellular fibrinogen binding protein or clumping factor A or B), are at least 3 and less than 27 amino acids in length, or are specific for bacteria, viruses, or cancer cells. Some antigenic domains of ligand/receptor specificity exchangers comprise a peptide having an antibody-binding region of a pathogen or toxin, are at least 5 and less than 35 amino acids in length, or contain peptides that are obtained from polio virus, TT virus, hepatitis B virus, and herpes simplex virus. More specificity exchangers concern glycoconjugate peptides comprising an HIV gp120 binding fragment of CD4 synthetically conjugated to gal α (1,3) gal β. (See e.g., U.S. Pat. Nos. 7,318,926; 7,335,359; and 7,332,166, all of which are hereby expressly incorporated by reference in their entireties). A better understanding of ways to use these specificity exchangers to provide effective therapy is needed.

### BRIEF SUMMARY OF THE INVENTION

Aspects of the invention concern the use of a specificity exchanger comprising a specificity domain that is less than 200 amino acids in length joined to at least one saccharide to induce an antibody-dependent cellular cytotoxicity response (ADCC) and/or neutralize viral infection. In some embodiments, the saccharide that is synthetically conjugated to the specificity domain is a Gal antigen, preferably, Gal α (1,3) Gal β and in other embodiments the saccharide is a blood group antigen. These specificity exchangers can be ligand/receptor specificity exchangers or antigen/antibody specificity exchangers. Although the saccharide can be directly joined to the specificity domain such that there is no antigenic domain or linker, some embodiments include an antigenic domain and/or linker in addition to the saccharide.

Preferred specificity exchangers are directed to HIV and the specificity domains of these embodiments can comprise a CD4, an HIV binding fragment of a CD4 or a CDR peptide (e.g., a sequence selected from the group consisting of SEQ. ID. No.1, SEQ. ID. No. 2, SEQ. ID. No. 3, SEQ. ID. No. 4, SEQ. ID. No. 5, SEQ. ID. No. 6, SEQ. ID. No. 7, SEQ. ID. No. 8, and SEQ. ID. No. 9) and said at least one saccharide is Gal α (1,3) Gal β or a blood group antigen. The specificity exchangers described above can have a specificity domain or antigenic domain that is less than 150, 100, 50, 35, 25, 15, 10, 9, 8, or 5 amino acids in length.

The specificity exchangers described herein can be used to target HIV-1 infected lymphocytes for ADCC activity and/or neutralization. That is, methods of redirecting anti-gal (a 1,3) gal β antibodies that are naturally present in a subject to HIV infected cells, thereby inducing ADCC activity or HIV neutralization; methods of treating or preventing HIV infection; and, methods of ameliorating a condition associated with using one or more of the specificity exchangers described herein are also embodiments.

Some embodiments describe a method of inducing an antibody dependent cellular cytotoxicity (ADCC) in a subject in need thereof, comprising identifying a subject in need of an ADCC response against HIV infected cells, wherein the subject has a CD4 cell count that allow safe induction of an ADCC response against CD4 infected cells, providing to the identified subject an effective amount of a glycoconjugate peptide comprising a binding fragment of a CD4 receptor for HIV gp120 synthetically conjugated to gal α (1,3) gal β, and measuring the reduction of HIV viral load in the subject. In some aspects, the subject is evaluated for presence of natural antibodies specific for gal α (1,3) gal β before administration of the glycoconjugate peptide.

In some aspects of the embodiment, the gal α (1,3) gal β is synthetically conjugated to the binding fragment of a CD4 receptor for HIV gp120 by attachment at one amino acid. In some aspects, the binding fragment of a CD4 receptor for HIV gp120 is less than 200 amino acids in length. In other aspects, the binding fragment of a CD4 receptor for HIV gp120 is less than 150 amino acids in length. In some aspects, the binding fragment of a CD4 receptor for HIV gp120 is less than 100 amino acids in length. In other aspects, the binding fragment of a CD4 receptor for HIV gp120 is less than 50 amino acids in length. In some aspects, the binding fragment of a CD4 receptor for HIV gp120 is less than 25 amino acids in length. In some aspects, the binding fragment of a CD4 receptor for HIV gp120 is less than or equal to 15 amino acids in length. In some aspects, the gal α (1,3) gal β is synthetically conjugated to a hydroxylated amino acid. In other aspects, the gal α (1,3) gal β is synthetically conjugated by an NH2-linkage. In still other aspects, the gal α (1,3) gal β is synthetically conjugated to the N-terminal end of the binding fragment of a CD4 receptor for HIV gp120.

In some aspects, the subject is a human.

Some embodiments further comprise measuring ADCC of said infected cells mediated by NK cells.

Some embodiments describe a method of neutralizing Human Immunodeficiency Virus (HIV) infection comprising identifying an HIV infected cell, and contacting the cell with a glycoconjugate peptide comprising a binding fragment of a CD4 receptor for HIV gp120 synthetically conjugated to gal α (1,3) gal β, and measuring neutralization of HIV.

In some aspects of the embodiment, the gal α (1,3) gal β is synthetically conjugated to the binding fragment of a CD4 receptor for HIV gp120 by attachment at one amino acid. In some aspects, the binding fragment of a CD4 receptor for HIV gp120 is less than 200 amino acids in length. In other aspects, the binding fragment of a CD4 receptor for HIV gp120 is less than 150 amino acids in length. In some aspects, the binding fragment of a CD4 receptor for HIV gp120 is less than 100 amino acids in length. In other aspects, the binding fragment of a CD4 receptor for HIV gp120 is less than 50 amino acids in length. In some aspects, the binding fragment of a CD4 receptor for HIV gp120 is less than 25 amino acids in length. In some aspects, the binding fragment of a CD4 receptor for HIV gp120 is less than or equal to 15 amino acids in length. In some aspects, the gal α (1,3) gal β is synthetically conjugated to a hydroxylated amino acid. In other aspects, the gal α (1,3) gal β is synthetically conjugated by an NH2-linkage. In still other aspects, the gal α (1,3) gal β is synthetically conjugated to the N-terminal end of the binding fragment of a CD4 receptor for HIV gp120.

Some embodiments describe a method of inducing an antibody dependent cellular cytotoxicity (ADCC) response against an HIV infected cell comprising indentifying an HIV infected cell, contacting the cell with an effective amount of a glycoconjugate peptide comprising a binding fragment of a CD4 receptor for HIV gp120 synthetically conjugated to gal α (1,3) gal β, and measuring an ADCC response against the HIV infected cell.

In some aspects of the embodiment, the measuring of the ADCC response comprises a measurement of cellular cytotoxicity mediated by NK cells.

In some aspects of the embodiment, the gal α (1,3) gal β is synthetically conjugated to the binding fragment of a CD4 receptor for HIV gp120 by attachment at one amino acid. In some aspects, the binding fragment of a CD4 receptor for HIV gp120 is less than 200 amino acids in length. In other aspects, the binding fragment of a CD4 receptor for HIV gp120 is less than 150 amino acids in length. In some aspects, the binding fragment of a CD4 receptor for HIV gp120 is less than 100 amino acids in length. In other aspects, the binding fragment of a CD4 receptor for HIV gp120 is less than 50 amino acids in length. In some aspects, the binding fragment of a CD4 receptor for HIV gp120 is less than 25 amino acids in length. In some aspects, the binding fragment of a CD4 receptor for HIV gp120 is less than or equal to 15 amino acids in length. In some aspects, the gal α (1,3) gal β is synthetically conjugated to a hydroxylated amino acid. In other aspects, the gal α (1,3) gal β is synthetically conjugated by an NH2-linkage. In still other aspects, the gal α (1,3) gal β is synthetically conjugated to the N-terminal end of the binding fragment of a CD4 receptor for HIV gp120.

In some aspects of the embodiment, the HIV infected cell is present in a human.

### BRIEF DESCRIPTION OF THE DRAWINGS

FIG. 1 illustrates a method that can be employed to artificially synthesize glycopeptide libraries.

FIG. 2 illustrates a method to artificially synthesize cyclic glycopeptides.

FIG. 3 illustrates the different reactivity of CD4 ligand/receptor specificity exchangers comprising glycosylated (gal-α-1,3 gal-β) or unglycosylated antigenic domains to four different human sera samples. The “X” axis shows the specificity exchangers provided at 5 μg/ml and the “Y” axis shows the OD at 405/650 after detection of antibodies bound.

FIG. 4 illustrates the inhibition of binding of CD4 ligand/receptor specificity exchangers comprising glycosylated (gal-α-1,3 gal-β) or unglycosylated antigenic domains in the presence of glycosylated (gal-α-1,3 gal-β) bovine serum albumin (BSA). The “X” axis shows the concentration of peptide and the “Y” axis shows the OD at 405/650 after detection of antibodies bound.

FIGS. 5 (A-F) illustrates an Elisa conducted for CD4 glycopeptides, wherein plates were coated with gp120 from LAI virus and peptide was added at shown concentrations. After incubation human serum was added at progressive dilutions and binding was assessed by a polyvalent anti-human antibody. As negative controls: an irrelevant peptide also coupled to the sugar and human serum depleted of the anti-gal-alpha 1,3-gal antibodies.

FIG. 6 illustrates a plaque assay in U87 cells in the presence of CD4 ligand/receptor specificity exchangers (also referred to as CD4 glycopeptides or CD4 glycocunjugated peptides).

FIG. 7 illustrates an infectivity assay after a single round of replication in TZMbl cells in the presence of CD4 glycopeptides.

FIGS. 8 (A-B) illustrates an infectivity assay conducted in H9 cells as determined by RT activity, wherein measurements were taken at 11 days post infection. In FIG. 5A human serum was added at a 10% dilution whereas in FIG. 8B human serum was added at a a 5% dilution.

FIG. 9 illustrates the effect of complement on neutralization, wherein 5 or 10% non-heat inactivated human serum was analyzed in the presence of the CD4 glycopeptides in both the plaque assay (left) and RT assay (right).

FIG. 10 illustrates an ADCC assay in the presence of the CD4 glycopeptides (10 μg/ml concentration plus 10% HS), wherein an anti-CD5 antibody was used to label chronically infected ACH2 cells (target cells) and freshly isolated NK cells were added to evaluate cellular cytotoxicity.

FIGS. 11 (A-E) illustrates binding of glycopeptide 4 to infected ACH2 cells using Alexa Fluor-conjugated isolectin B4.

FIGS. 12 (A-M) illustrates a single round infectivity assay on TZM-bl cells, a syncytia inhibition assay on U87 cells and a multiple round infectivity assay on H9 cells.

FIGS. 13 (A-C) illustrates dot plots of chronically infected ACH2 cells pre-treated with glycopeptides (top and bottom rows of FIGS. 13A-C) and inactivated human serum (top row of FIG. 13A-C) prior to the addition of freshly isolated NK cells. The figures listed in the upper-right quadrant represent the percentage of ACH2 cells that positively stained for both dyes, i.e. the level of cytotoxicity.

### DETAILED DESCRIPTION OF THE INVENTION

It has been discovered that specificity exchangers comprising an HIV binding fragment of CD4 synthetically conjugated to a gal α (1,3) gal β react strongly to antibodies that are naturally present in a subject and thereby promote the redirection of said antibodies to HIV-1 infected cells, induce antibody dependent cell cytotoxicity (ADCC) and promote HIV-1 neutralization. Aspects of the invention concern methods of inducing HIV-1 neutralization and an ADCC response by providing an effective amount of a glycoconjugate peptide comprising an HIV binding fragment of CD4 synthetically conjugated to gal α (1,3) gal β or another blood group antigen (e.g., Lewis X-BSA, 2′-Fucosyllactose-BSA (2′FL-BSA), Lacto-N-fucopentaose II-BSA, Lacto-N-fucopentaose III-BSA, Lacto-N-fucopentaose I-BSA (LNFPI-BSA), Lacto-N-difucohexaose I-BSA (LNDFHI-BSA), Blood Group A-BSA, Blood Group B-BSA, Globotriose-HSA, Galα1-4Galb1-4Glc-HSA). Optimally, the method above includes a step of measuring the ADCC response and/or HIV-1 neutralization, which can be accomplished by conventional methods, such as described herein (e.g., measuring the number of HIV infected lymphocytes before, during or after providing the glycoconjugated peptide or measuring proliferation of HIV by RTPCR or immunoassay). In some embodiments, the subject (e.g., human patient) is identified to receive a treatment that induces an ADCC response and/or HIV neutralization and an amount of glycoconjugate peptide, as described herein, is provided to the identified subject in an amount to induce said ADCC response and/or HIV neutralization. Identification of subjects to receive a treatment that induces an ADCC response and/or HIV neutralization using one of the glycoconjugate peptides described herein may be important since a reduction in HIV-1 infected lymphocytes may require hospitalization or alternative therapies and may not be suitable for patients at some stages of HIV infection. Identification of subjects to receive a treatment that induces an ADCC response and/or HIV neutralization can be made by chemical evaluation (e.g., white blood cell count) or diagnostic assays as known in the field.

The specificity exchangers that can be used with the embodied methods can comprise a specificity domain that is desirably between at least 3-200 amino acids, preferably between at least 5-100 amino acids, more preferably between 8-50 amino acids, and still more preferably between 10-25 amino acids and is preferably an HIV binding fragment of CD4. These specificity domains are joined to one or more sugars (e.g., a glycosylation domain having one or more gal-α-1-3 gal β sugars or a blood group antigen, such as Lewis X-BSA, 2′-Fucosyllactose-BSA (2′FL-BSA), Lacto-N-fucopentaose II-BSA, Lacto-N-fucopentaose III-BSA, Lacto-N-fucopentaose I-BSA (LNFPI-BSA), Lacto-N-difucohexaose I-BSA (LNDFHI-BSA), Blood Group A-BSA, Blood Group B-BSA, Globotriose-HSA, Galα1-4Galb1-4Glc-HSA) that is itself an antigenic domain that interacts with antibodies that are naturally present in a subject.

The interaction between gp120 and its receptor the CD4 molecule is highly conserved and involves only a limited amount of residues. The envelope glycoprotein binds to 22 residues on the D1 region of the CD4 receptor located between amino acids 25 to 64 and the atomic interactions are well characterized. Most of the contact is established by amino acids of the CDR2 like region with Phe 43 and Arg 59 playing prominent roles. Molecules that mimic the CD4 receptor will not induce the selection of mutants since any changes in the binding region would be deleterious for the infectivity of the virus.

Natural antibodies play a prominent role in the first line of defense against many viral and bacterial infections, providing a link between the innate and adaptive immune response. They have been shown to be prominent in the antibody-mediated opsonization of particles by macrophages and in the induction of the classical cascade of the complement. Moreover, these antibodies can trigger the activation of NK cells via CD16 recognition of the Fc fraction of the antibody and induce an antibody-dependent cellular cytotoxicity (ADCC). In human serum approximately 1 to 8% of the total IgM and 1 to 2.4% of the total IgG recognize the epitope gal(α1,3)gal, which is part of a penta-saccharide present mainly on endothelial cells of all mammals except humans and old world monkeys. Thus, aspects of the invention concern the temporary redirection of this preexisting antibody pool to a new antigen, which rapidly would increase the pool of biologically active antibodies with a pre-determined specificity as an alternative to administering a monoclonal antibody.

In preferred embodiments, the specificity domain comprises a CD4 peptide that binds to HIV (e.g., a ligand or portion of an antibody) that is between 3 and 150 amino acids in length (e.g., 3, 5, 8, 9, 10, 12, 14, 17, 20, 22, 25, 28, 30, 35, 37, 40, 45, 50, 55, 60, 65, 70, 75, 80, 85, 90, 100, 125, or 150 amino acids). The antigenic domain, which is joined to said specificity domain, can be 0, 3, 5, 8, 9, 10, 12, 14, 17, 20, 22, 25, 28, 30, 35, 37, 40, 45, 50, 55, 60, 65, 70, 75, 80, 85, 90, 100, 125, or 150 amino acids in length and can comprise at least one saccharide, a blood group antigen preferably, Gal α (1,3) Gal β. That is, in some embodiments the at least one saccharide, preferably, Gal α (1,3) Gal β, is directly joined to the specificity domain. Embodiments include, for example, Seq. Id. No. 1, 2, 3, 4, 5, 6, 7, 8, or 9 (Table 1).

TABLE 1
HIV specific ligand/receptor specificity domains

| Glycosylated | | | |
|---|---|---|---|
| Sp.exchanger | Glyconentide | | |
| Name | (GP) | Sequence | Identifier |
| Gal-CDR | | DCDLIYYDYEEDYYFDY | (Seq. Id. No.1) |
| Gal-CD4-1 | | DQFHWKNSNQIKILGN | (Seq. Id. No.2) |
| Gal-CD4-4 | | DQGSFLTKGPSKLNDR | (Seq. Id. No.3) |
| Gal-CD4-100 | GP1 | QFHWKNSNQIKILGN | (Seq. Id. No.4) |
| Gal-CD4-101 | GP2 | NSNQIKILGNQGSFL | (Seq. Id. No.5) |
| Gal-CD4-102 | GP3 | KILGNQGSFLTKGPS | (Seq. Id. No.6) |
| Gal-CD4-103 | GP4 | QGSFLTKGPSKLNDR | (Seq. Id. No.7) |
| Gal-CD4-104 | GPS | TKGPSKLNDRADSRR | (Seq. Id. No.8) |
| Gal-CD4-105 | GP6 | KLNDRADSRRSLWDQ | (Seq. Id. No.9) |

The next section describes some of the antigenic domains that can be used with the specificity exchangers described herein.

Antigenic Domains

The diversity of antigenic domains that can be used with the glycoconjugated peptides described herein is quite large. In some embodiments, these antigenic domains serve as a linker for the saccharides and/or a site to which other antibodies are directed. Desirably, the antigenic domains used with the specificity exchangers are peptides obtained from surface proteins or exposed proteins from bacteria, fungi, plants, molds, viruses, cancer cells, and toxins. It is also desired that the antigenic domains comprise a peptide sequence that is rapidly recognized as non-self by existing antibodies in a subject, preferably by virtue of naturally acquired immunity or vaccination. For example, many people are immunized against childhood diseases including, but not limited to, small pox, measles, mumps, rubella, and polio. Thus, antibodies to epitopes on these pathogens can be produced by an immunized person. Desirable antigenic domains have a peptide that contains one or more epitopes that is recognized by antibodies in the subject that are present in the subject to respond to pathogens such as small pox, measles, mumps, rubella, herpes, hepatitis, and polio.

Some embodiments, however, have antigenic domains that interact with an antibody that has been administered to the subject. For example, an antibody that interacts with an antigenic domain on a specificity exchanger can be co-administered with the specificity exchanger. Further, an antibody that interacts with a specificity exchanger may not normally exist in a subject but the subject has acquired the antibody by introduction of a biologic material or antigen (e.g., serum, blood, or tissue) so as to generate a high titer of antibodies in the subject. For example, subjects that undergo blood transfusion acquire numerous antibodies, some of which can interact with an antigenic domain of a specificity exchanger. Some preferred antigenic domains for use in a specificity exchanger also comprise viral epitopes or peptides obtained from pathogens such as the herpes simplex virus, hepatitis B virus, TT virus, and the poliovirus.

Preferably, the antigenic domains comprise an epitope or peptide obtained from a pathogen or toxin that is recognized by a “high-titer antibody.” The term “high-titer antibody” as used herein, refers to an antibody that has high affinity for an antigen (e.g., an epitope on an antigenic domain). For example, in a solid-phase enzyme linked immunosorbent assay (ELISA), a high titer antibody corresponds to an antibody present in a serum sample that remains positive in the assay after a dilution of the serum to approximately the range of 1:100-1:1000 in an appropriate dilution buffer. Other dilution ranges include 1:200-1:1000, 1:200-1:900, 1:300-1:900, 1:300-1:800, 1:400-1:800, 1:400-1:700, 1:400-1:600, and the like. In certain embodiments, the ratio between the serum and dilution buffer is approximately: 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, 1:1000. Epitopes or peptides of a pathogen that can be included in an antigenic domain of a specificity exchanger include the epitopes or peptide sequences disclosed in Swedish Pat No. 9901601-6; U.S. Pat. No. 5,869,232; Mol. Immunol 28: 719-726 (1991); and J. Med Virol. 33:248-252 (1991); all which are herein expressly incorporated by reference in their entireties.

The specificity exchangers described herein are not required to have antigenic peptide domains, however. In some embodiments, a sugar, a plurality of sugars, a glycosylation region or a glycosylation domain is itself the epitope(s) to which antibodies that are naturally present in a subject are directed. That is, some embodiments are specificity exchangers that comprise a specificity domain (e.g., an HIV gp120 binding fragment of CD4) that is joined to a sugar, a plurality of sugars, a glycosylation region, or a glycosylation domain with or without a peptide linker but lacking an antigenic peptide or epitope obtained from a pathogen or toxin. In this manner, glycosylated specificity domains are also referred to as glycosylated specificity exchangers or glycoconjugate peptides, wherein the sugar, plurality of sugars, glycosylation region or glycosylation domain is itself the antigenic domain. The next section describes glycosylated specificity exchangers in greater detail.

Specificity Exchangers Comprising Saccharides and Glycoconjugates

Generally, the glycosylated specificity exchangers (i.e., glycoconjugate peptides) comprise a specificity domain that is at least 3 and less than or equal to 200 amino acids in length joined to a plurality of saccharides that, together with the peptide backbone or by itself, react with high titer antibodies that are naturally present in a human. Preferably, the glycosylation domain or region contains blood group sugars that are xenoactive antigens (e.g., blood group sugars that are the basis for hyperactute rejection of xenografts or transplantations).

In some embodiments, for example, the specificity exchangers comprise a specificity domain that is between or at least and/or less than or equal to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 amino acids in length, preferably, an HIV gp120 binding fragment of CD4 and said specificity domain can be joined to an antigenic domain (e.g., a peptide backbone) that is between or at least and/or less than or equal to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 amino acids in length, wherein said antigenic domain or specificity domain or both comprise a plurality of saccharides, a blood group antigen or preferably gal α(1,3) gal. Other embodiments comprise a specificity domain that is between or at least and/or less than or equal to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 amino acids in length, preferably, an HIV gp120 binding fragment of CD4 and said specificity domain is joined to a plurality of saccharides (with or without a peptide linker and with or without a peptide or epitope of a pathogen or with or without an antigenic domain) a blood group antigen, or preferably gal α(1,3) galβ. Depending on the embodiment, the “plurality of saccharides” can include at least 2 and 10,000 or more sugar units. In some embodiments, for example, between or at least and/or less than or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2250, 2500, 2750, 3000, 3250, 3500, 3750, 4000, 4250, 4500, 4750, 5000, 5250, 5500, 5750, 6000, 7000, 8000, 9000, 10,000 or more sugar units are joined to the specificity domain either directly or indirectly (e.g., through a support such as the peptide backbone of a linker an antigenic domain comprising a peptide or epitope of a pathogen).

The term “saccharide” is intended to be construed broadly so as to non-exclusively encompass monosaccharides, disaccharides, polysaccharides (glycans), oligosaccharides, and other similar compounds. The term “glycoconjugate” is also to be construed broadly, and generally refers to an organic compound consisting of one or more carbohydrate units (e.g., a saccharide) joined to a support.

In several embodiments, the specificity domain is joined to a support to which a plurality of saccharides and/or a glycoconjugate is also joined. A “support” can be a peptide backbone, (e.g., an antigenic domain, as described above), a protein, a resin, or any macromolecular structure that can be used to join or immobilize a saccharide or a specificity domain. The saccharides and specificity domains can be joined to inorganic supports, such as silicon oxide material (e.g., silica gel, zeolite, diatomaceous earth or aminated glass) by, for example, a covalent linkage through a hydroxy, carboxy, or amino group and a reactive group on the support. In some embodiments, the support has a hydrophobic surface that interacts with a portion of the specificity domain and/or saccharide or saccharide conjugate (e.g., glycolipid) by a hydrophobic non-covalent interaction. In some cases, the hydrophobic surface of the support is a polymer such as plastic or any other polymer in which hydrophobic groups have been linked such as polystyrene, polyethylene or polyvinyl.

Additionally, supports such as proteins and oligo/polysaccarides (e.g., cellulose, starch, glycogen, chitosane or aminated sepharose) can be used by exploiting reactive groups on the specificity domains or saccharides, such as a hydroxy or an amino group, to join to a reactive group on the support so as to create the covalent bond. Still more supports containing other reactive groups that are chemically activated so as to attach the saccharides and specificity domains can be used (e.g., cyanogen bromide activated matrices, epoxy activated matrices, thio and thiopropyl gels, nitrophenyl chloroformate and N-hydroxy succinimide chlorformate linkages, or oxirane acrylic supports).

The insertion of linkers (e.g., “λ linkers” engineered to resemble the flexible regions of λ phage) of an appropriate length between the specificity domain and/or the plurality of saccharides and the support are also contemplated so as to encourage greater flexibility and overcome any steric hindrance that can be encountered. The determination of an appropriate length of linker that allows for optimal binding can be found by screening the attached molecule with varying linkers in the characterization assays detailed herein.

Preferred embodiments include specificity exchangers that comprise glycoconjugates and support-bound saccharides that are commonly referred to as glycoproteins, proteoglycans, glycopeptides, peptidoglycans, glyco-amino-acids, glycosyl-amino-acids, glycolipids, and related compounds. The glycoproteins that can be used with an embodiment described herein include compounds that contain a carbohydrate and a protein. The carbohydrate may be a monosaccharide, disaccharide(s), oligosaccharide(s), polysaccharide(s), their derivatives (e.g., sulfo- or phospho-substituted), and other similar compounds. There are two major classes of glycoproteins that can be used, O-linked glycans and the N-linked glycans. N-linked glycoproteins contain an N-acetylglucosamine residue linked to the amide nitrogen of an asparagine residue in the protein. The most common O-linkage involves a terminal N-acetylgalactosamine residue in the oligosaccharide linked to a serine or threonine residue of the protein. While specificity exchangers that comprise a glycoprotein can include one, a few, or many carbohydrate units, some embodiments comprise a proteoglycan, a subclass of glycoproteins that are polysaccharides that contain amino sugars.

The glycopeptides that can be used with some of the embodiments described herein include compounds having a carbohydrate linked to an oligopeptide composed of L- and/or D-amino acids. The peptidoglycans that can be used comprise a glycosaminoglycan formed by alternating residues of D-glucosamine and either muramic acid {2-amino-3-O—[(R)-1-carboxyethyl]-2-deoxy-D-glucose} or L-talosaminuronic acid (2-amino-2-deoxy-L-taluronic acid), which are usually N-acetylated or N-glycosylated.

The glyco-amino-acids that can be used with the embodiments described herein comprise a saccharide attached to a single amino acid, whereas the glycosyl-amino-acids that can be used include compounds comprising a saccharide linked through a glycosyl linkage (O—, N— or S—) to an amino acid. (The hyphens are used to avoid implying that the carbohydrate is necessarily linked to the amino group.) In some embodiments, the antigenic domain comprises a glycolipid, which is a compound comprising one or more monosaccharide residues bound by a glycosidic linkage to a hydrophobic moiety such as an acylglycerol, a sphingoid, a ceramide (N-acylsphingoid) or a prenyl phosphate, for example. Some of the specificity exchangers described herein can also comprise a glycoconjugate (e.g., lectins).

Preferred embodiments, however, include specificity exchangers that comprise human proteins or glycoconjugates that are commonly referred to as blood group antigens. These antigens are generally surface markers located on the outside of red blood cell membranes. Most of these surface markers are proteins, however, some are carbohydrates attached to lipids or proteins. Structurally, the blood group determinants that can be used with the embodiments described herein fall into two basic categories known as type I and type II. Type I comprises a backbone comprised of a galactose 1-3 β linked to N-acetyl glucosamine while type II comprises, instead, a 1-4 β linkage between the same building blocks (cf N-acetyl lactosamine). The position and extent of a-fucosylation of these backbone structures gives rise to the Lewis-type and H-type specificities. Thus, monofucosylation at the C₄-hydroxyl of the N-acetyl glucosamine (Type I series) constitutes the Le^{a} type, whereas fucosylation of the C₃-hydroxyl of this sugar (Type II series) constitutes the Le^{x} determinant. Additional fucosylation of Le^{a} and Le^{x} types at the C₂,-hydroxyl of the galactose sector specifies the Le^{b} and Le^{y} types, respectively.

The presence of an a-monofucosyl branch, solely at the C₂,-hydroxyl in the galactose moiety in the backbone, constitutes the H-type specifity (Types I and TI). Further permutation of the H-types by substitution of a-linked galactose or a-linked N-acetylgalactosamine at its ,-hydroxyl group provides the molecular basis of the familiar serological blood group classifications A, B, and O. (See e.g., Lowe, J. B., The Molecular Basis of Blood Diseases, Stamatoyannopoulos, et. al., eds., W.B. Saunders Co., Philadelphia, Pa., 1994, 293., herein expressly incorporated by reference in its entirety.)

By first determining a patient's particular set of blood group antigens, one can select a specificity exchanger comprising one or more blood group antigens that are outside of the repertoire of the patient so as to generate a potent response to the antigenic domain of the specificity exchanger in the patient and thereby redirect the antibodies present in the patient to the pathogen that is specific for the specificity domain of the specificity exchanger. Accordingly, specificity exchangers that are specific for several different pathogens can be made to have antigenic domains that comprise many different combinations of blood group antigens so that a potent immune response can be obtained in any particular individual. The next section describes the manufacture of specificity exchangers comprising saccharides and glycoconjugates, in particular blood group antigens, in greater detail.

Making Specificity Exchangers that Comprise Saccharides and Glycoconjugates Several issues merit consideration in contemplating the synthesis of such blood group substances and their neoglycoconjugates, however. For purposes of synthetic economy it is helpful to gain relief from elaborate protecting group manipulations common to traditional syntheses of complex branched carbohydrates. Another issue involves fashioning a determinant linked to a protein carrier. In crafting such constructs, it may be beneficial to incorporate appropriate spacer units between the carbohydrate determinant and the carrier. (See e.g., Stroud, M. R., et al., Biochemistry, 1994, 33, 10672; Yuen, C.-T., et al., J. Biochem., 1994, 269, 1595; and Stroud, M. R., et al, J. Biol. Chem., 1991, 266, 8439., all of which are herein expressly incorporated by reference in entirities). TABLE 2 provides a non-exclusive list of blood group antigens that can be joined to or incorporated in a specificity exchanger.

TABLE 2

| | Blood group carrier or | | SWISS-PROT | |
|---|---|---|---|---|
| | effector protein name | | cross | Antigen |
| | system | Gene name | reference | names |
| ABO | Fucosylglycoprotein alpha-n- | Gene: ABO | BGAT_HUMAN | Antigens: A/B |
| | acetylgalactosaminyltransferase | | (P16442) | |
| | (EC 2.4.1.40) (Histo- | | | |
| | blood group A transferase)/ | | | |
| | Fucosylglycoprotein 3-alpha- | | | |
| | galactosyltransferase (EC | | | |
| | 2.4.1.37) (Histo-blood group | | | |
| | B transferase) | | | |
| Chido/ | Complement C4 | Gene: C4A and C4B | CO4_HUMAN | Antigens: Ch1 |
| Rodgers | | | (P01028) | to Ch6, WH, |
| | | | | Rg1, Rg2 |
| Colton | Aquaporin-CHIP (Aquaporin | Gene: AQP1; | AQP1_HUMAN | Antigens: |
| | 1). | CHIP28 | (P29972) | Co(a/b) |
| Cromer | Complement decay- | Gene: DAF; CD55 | DAF_HUMAN | Antigens: |
| | accelerating factor (Antigen | | (P08174) | Cr(a), Dr(a), |
| | CD55). | | | Es(a), |
| | | | | Tc(a/b/c), |
| | | | | Wd(a), |
| | | | | WES(a/b), IFC, |
| | | | | UMC |
| Diego | Band 3 anion transport | Gene: SLC4A1; | B3AT_HUMAN | Antigens: |
| | protein (Anion exchange | AE1; EPB3 | (P02730) | Di(a/b), |
| | protein 1) (AE 1). | | | Wr(a/b), |
| | | | | Wd(a), Rb(a), |
| | | | | WARR |
| Dombrock | Dombrock glycoprotein. | Gene: DO | Not yet | Antigens: |
| | | | identified | Do(a/b), |
| | | | | Gy(a), Hy, |
| | | | | Jo(A) |
| Duffy | Duffy antigen (Fy | Gene: FY; GPD; | DUFF_HUMAN | Antigens: |
| | glycoprotein) (Glycoprotein | DARC | (Q16570) | Fy(a/b) |
| | D) (GpFy). | | | |
| Gerbich | Glycophorin C (PAS-2′) | Gene: GYPC; GPC | GLPC_HUMAN | Antigens: |
| | (Glycoprotein beta) | | (P04921) | An(a), Dh(A), |
| | (Glycoconnectin) | | | Ls(a), Wb |
| | (Sialoglycoprotein D) | | | |
| | (Glycophorin D) (GpD). | | | |
| Hh | Galactoside 2-L- | Gene: FUT1 | FUT1_HUMAN | |
| | fucosyltransferase 1 (EC | | (P19526) | |
| | 2.4.1.69) (Alpha(1,2)Ft 1) | | | |
| | (Fucosyltransferase 1). | | | |
| | Galactoside 2-L- | Gene: FUT2 | FUT2_HUMAN | Antigens: H/h, |
| | fucosyltransferase 2 (EC | | (Q10981) | Se/se |
| | 2.4.1.69) (Alpha(1,2)Ft 2) | | | |
| | (Fucosyltransferase 2) | | | |
| | (Secretor factor). | | | |
| Indian | CD44 antigen (Phagocytic | Gene: CD44; LHR | CD44_HUMAN | Antigens: |
| | glycoprotein I) (PGP-1) | | (P16070) | In(a/b) |
| | (Hutch-I) (Extracellular | | | |
| | matrix receptor-III) (ECMR- | | | |
| | III) (Hermes antigen) | | | |
| | (Hyaluronate receptor) | | | |
| | (Heparan sulfate | | | |
| | proteoglycan) (Epican). | | | |
| Kell | Kell blood group glycoprotein | Gene: KEL | KELL_HUMAN | Antigens: K/k, |
| | (EC 3.4.24.—). | | (P23276) | Kp(a/b/c), |
| | | | | Js(a/b), Ul(a), |
| | | | | KEL11/17, |
| | | | | KEL14/24 |
| Kidd | Urea transporter, | Gene: SLC14A1; | UT1_HUMAN | Antigens: |
| | erythrocyte. | UT1; HUT11; UTE; | (Q13336) | Jk(a/b) |
| | | JK; RACH1 | | |
| Knops | Complement receptor type 1 | Gene: CR1; C3BR | CR1_HUMAN | Antigens: |
| | (C3b/C4b receptor) (Antigen | | (P17927) | Kn(a/b), |
| | CD35). | | | McC(a), Sl(a), |
| | | | | Yk(a) |
| Kx | Membrane transport protein | Gene: XK | XK_HUMAN | |
| | XK (Kx antigen). | | (P51811) | |
| Landsteiner- | Landsteiner-Wiener blood | Gene: LW | LW_HUMAN | Antigens: |
| Wiener | group glycoprotein. | | (Q14773) | Lw(a/b) |
| Lewis | Galactoside 3(4)-L- | Gene: FUT3; LE | FUT3_HUMAN | Antigens: |
| | fucosyltransferase (EC | | (P21217) | Le(a/b) |
| | 2.4.1.65) (Fucosyltransferase | | | |
| | 3) (FUCT-III). | | | |
| Lutheran | Lutheran blood group | Gene: LU; BCAM; | LU_HUMAN | Antigens: |
| | glycoprotein (B-CAM cell | MSK19 | (P50895) | Lu(a/b), |
| | surface glycoprotein) | | | Au(a/b), LU6 |
| | (Auberger B antigen) | | | to LU20 |
| | (F8/G253 antigen). | | | |
| MNS | Glycophorin A (PAS-2) | Gene: GYPA; GPA | GLPA_HUMAN | Antigens: M/N, |
| | (Sialoglycoprotein alpha) | | (P02724) | S/s, U, He, |
| | (MN sialoglycoprotein). | | | Mi(a), M(c), |
| | Glycophorin B (PAS-3) | Gene: GYPB; GPB | GLPB_HUMAN | Vw, Mur, M(g), |
| | (Sialoglycoprotein delta) | | (P06028) | Vr, M(e), |
| | (SS-active sialoglycoprotein). | | | Mt(a), St(a), |
| | | | | Ri(a), Cl(a), |
| | | | | Ny(a), Hut, Hil, |
| | | | | M(v), Far, Mit, |
| | | | | Dantu, Hop, |
| | | | | Nob, En(a), |
| | | | | ENKT, etc. |
| P | A yet undefined | Gene: P1 | Not yet | Antigens: P1 |
| | galactoyltransferase. | | identified | |
| Rh | Blood group RH(CE) | Gene: RHCE; RHC; | RHCE_HUMAN | Antigens: C/c, |
| | polypeptide (Rhesus C/E | RHE | (P18577) | E/e, D, f, C(e), |
| | antigens) (RHPI). | | | C(w), C(x), V, |
| | Blood group RH(D) | Gene: RHD | RHD_HUMAN | E(w), G, Tar, |
| | polypeptide (Rhesus D | | (Q02161) | VS, D(w), cE, |
| | antigen) (RHPII). | | | etc. |
| Scianna | Scianna glycoprotein. | Gene: SA | Not yet | Antigens: |
| | | | identified | Sc(1/2), Sc3 |
| Xg | Xg glycoprotein (Protein | Gene: XG; PBDX | XG_HUMAN | Antigens: |
| | PBDX). | | (P55808) | Xg(a) |
| Yt | Acetylcholinesterase (EC | Gene: ACHE | ACES_HUMAN | Antigens: |
| | 3.1.1.7). | | (P22303) | Yt(a/b) |

Additional blood groups can include Lewis X-BSA, 2′-Fucosyllactose-BSA (2′FL-BSA), Lacto-N-fucopentaose II-BSA, Lacto-N-fucopentaose III-BSA, Lacto-N-fucopentaose I-BSA (LNFPI-BSA), Lacto-N-difucohexaose I-BSA (LNDFHI-BSA), Blood Group A-BSA, Blood Group B-BSA, Globotriose-HSA, Galα1-4Galb1-4Glc-HSA, and the like.

While blood group antigens have been discussed in detail, it is important to point out that any saccharide or glycoconjugate can be included in the antigenic domain of the specificity exchangers described herein. Antigenic saccharides and glycoconjugates are well known in the art and are readily available from a commercial supplier such as V-Labs, Inc. (Covington, La.). Saccharides and glycoconjugates can also be synthesized using conventional techniques (as will be described in more detail). Potential saccharides and glycoconjugates that can be used herein can be derived from pathogens, including bacteria, viruses (e.g., L, M, and S glycoproteins from HBV, and gp160, gp120 and gp41 from HIV), protozoan, and fungi, cancer cells, toxins, cells affected by autoimmune diseases such as lupus, multiple sclerosis, rheumatoid arthritis, diabetes, psoriasis, Graves disease and the like.

Specific core structure neoglycoproteins that can be used in the antigenic domains described herein include: N-Acetyllactosamine-BSA (3-atom spacer), N-Acetyllactosamine-BSA (14-atom spacer), α1-3,α1-6 Mannotriose-BSA (14-atom spacer) and the like. Monosaccharide neoglycoproteins that can be used in the antigenic domains described herein include: N-Acetylglucosamine-BSA (14-atom spacer), N-Acetylgalactosamine-BSA (14-atom spacer), and the like. Tumor antigen neoglycoproteins that can be used in the antigenic domains described herein include: T-Antigen-HSA Galβ1-3GalNAc-HSA (3-atom spacer), Tn-Antigen-HSA GalNAcal-O-(Ser-N-Ac-CO)-Spacer-NH—HSA, and the like. Sialyated neoglycoproteins that can be used in the antigenic domains described herein include: 3′ Sialyl-N-acetyllactosamine-BSA (3-atom spacer), 3′-Sialyl-N-acetyllactosamine-BSA (14-atom spacer), 3′-Sialyl Lewis X-BSA (3-atom spacer), 3′-Sialyl Lewis^{x}-HSA (3-atom spacer), 3′-Sialyl-3-Fucosyl-Lactose-BSA (3-atom spacer), 3′-Sialyl Lewis^{x}-BSA (14-atom spacer), and the like.

In certain embodiments, the antigenic domain can include Gal α (1,3) Gal β(gal antigen), a carbohydrate antigen. The gal antigen is produced in large amounts on the cells of pigs, mice and New World monkeys by the glycosylation enzyme galactosyltransferase (α(1,3)GT). Galactosyltransferase is active in the Golgi apparatus of cells and transfers galactose from the sugar-donor uridine diphosphate galactose (UDP-galactose) to the acceptor N-acetyllactosamine residue on carbohydrate chains of glycolipids and glycoproteins, to form gal antigen.

The gal antigen is completely absent in humans, apes and Old World monkeys because their genes encoding a (1,3) GT have become inactivated in the course of evolution. (Xing et al. 01-2-x1 Cell Research 11(2): 116-124 (2001), herein expressly incorporated by reference in its entirety.) Since humans and Old World primates lack the gal antigen, they are not immunotolerant to it and produce anti-gal antigen antibodies (anti-Gal) throughout life in response to antigenic stimulation by gastrointestinal bacteria. (Id.) It has been estimated that as many as 1% of circulating B cells are capable of producing these antibodies. (Id.) The binding of anti-Gal to gal antigens expressed on glycolipids and glycoproteins on the surface of endothelial cells in donor organs leads to activation of the complement cascade and hyperacute rejection, and also plays an important role in occurrence of complement-independent delayed xenograft rejection. (Id.) Accordingly, the gal antigen has the ability to generate a potent immune response.

In certain embodiments the gal antigen to be joined or incorporated into a specificity exchanger is selected from gal α (1,3) gal series neoglycoproteins and can include: Galα1-3Gal-BSA (3-atom spacer), Galα1-3Gal-BSA (14-atom spacer), Galα1-3Gal-HSA (3-atom spacer), Galα1-3Gal-HSA (14-atom spacer), Galα1-3Galβ1-4GlcNAc-BSA (3-atom spacer), Galα1-3Galβ1-4GlcNAc-BSA (14-atom spacer), Galα1-3Galβ1-4GlcNAc-HSA (3-atom spacer), Galα1-3Galβ1-4GlcNAc-HSA (14-atom spacer), Galili Pentasaccharide-BSA (3-atom spacer), and the like. In other embodiments the gal antigen can be selected from gal α(1,3) gal analogue neoglycoproteins, including Galα1-3Galβ1-4Glc-BSA (3-atom spacer), Galα1-3Galβ1-4Glc-HSA (3-atom spacer), Galα1-3Galβ1-3GlcNAc-BSA (3-atom spacer), Galα1-3Galβ1-3GlcNAc-HSA (3-atom spacer), Galα1-3 Galβ1-4(3-deoxyGlcNAc)-HSA (3-atom spacer), Galα1-3Galβ1-4(6-deoxyGlcNAc)-HSA, and the like.

Danishefsky, et al., discloses several antigenic saccharides and glycoconjugates, and methods of synthesizing said compounds. (See U.S. Pat. No. 6,303,120, herein expressly incorporated by reference in its entirety). Specifically, this disclosure herein provides a method of synthesizing Le^{y}-related antigens as well as artificial protein-conjugates of the oligosaccharide. In certain embodiments, these antigens contain a novel array of features including the α-linkage between the B and the C entities, as well as the β-linked ring D gal-NAc residue. (For the synthesis of a related structure (SSEA-3), which lacks the fucose residue see: Nunomura, S.; Ogawa, T., Tetrahedron Lett., 1988, 29, 5681-5684., herein expressly incorporated by reference in its entirety.) In general, the methods described in U.S. Pat. No. 6,303,120, herein expressly incorporated by reference in its entirety, can be used or modified so as to join or incorporate the saccharides or glycoconjugates described herein with a specificity exchanger.

A major obstacle in the field of glycobiology is access to pure, chemically well defined complex carbohydrates and glycoconjugates. (See Randell, Karla D., et al., High-throughput Chemistry toward Complex Carbohydrates and Carbohydrate-like Compounds, National Research Council of Canada, publication no. 43876, Feb. 13, 2001, herein expressly incorporated by reference in its entirety). Unlike nucleic acids and polypeptides, these are non-linear molecules and the carbohydrate moieties present tremendous challenges in developing their total syntheses. (Id.) These polyhydroxy compounds contain an array of monosaccharide units and have a variety of glycosidic linkages between them. (Id.) Each glycosidic linkage can exist in the α- or β-anomeric configuration. (Id,) Therefore, carbohydrate syntheses can require many orthogonal protection-deprotection schemes and involve difficult glycosyl coupling reactions. (Id.) Recently, efforts have been made to develop automated syntheses of complex carbohydrates. (Id.)

While vastly more complicated than the techniques for synthesizing polynucleotides and polypeptides, techniques for synthesizing saccharides and glycoconjugates are known in the art. These techniques are discussed in the sections that follow as they fail into enzyme-based approaches, cell-based approaches, and chemical synthesis-based approaches.

Enzyme Synthesis

Different methods for synthesizing saccharides and glycoconjugates described herein can be found in U.S. Pat. No. 6,046,040, issued to Nishiguchi et al. (2000), which is hereby expressly incorporated by reference in its entirety. Specifically this patent discloses using enzyme-catalyzed in vitro reactions to synthesize saccharides and glycoconjugates. See also Toone et al., Tetrahedron Reports (1990) (45)17:5365-5422. Enzymatic approaches have been gaining popularity for the synthesis of saccharides and glycoconjugates in part because enzymes feature exquisite stereo- and regioselectivity and catalyze the reaction under very mild conditions. Extensive protection-deprotection schemes are thus unnecessary, and the control of anomeric configuration is simplified.

To produce some of the specificity exchangers described herein, the following enzymes may be used: saccarglycosyltransferases, glycosidases, glycosyl hydrolases or glycosyltransferases. Glycosyltransferases regulate the biosynthesis of carbohydrate antigens in cells and are responsible for the addition of carbohydrates to the oligosaccharide chain on glycolipids and glycoproteins in a sequential manner. Glycosyltransferases catalyze the addition of activated sugars, in a stepwise fashion, to a protein or lipid or to the non-reducing end of a growing oligosaccharide. Typically a relatively large number of glycosyltransferases are used to synthesize carbohydrates. Each NDP-sugar residue requires a distinct class of glycosyltransferase and each of the more than one hundred glycosyltransferases identified to date appears to catalyze the formation of a unique glycosidic linkage.

According to one enzyme-catalsyed method of synthesis, saccharides are synthesized using a solid phase method that utilizes glycal (Danishefsky et al, Science, 260, 1307 (1993)). This method includes (i) binding a glycal to a polystyrene-divinylbenzene copolymer via a diphenylsilyl group to allow reaction between the glycal and 3,3-dimethyldioxirane, that converts glycal to a 1,2-anhydrosugar, and (ii) using this anhydrosugar as a sugar donor, reaction with a different glycal suitably protected to form a glycoside glycal, and these steps are repeated. According to this method, a new glycosidic linkage is stereoselectively formed.

A solid phase method of sugar chain synthesis can also be used to generate saccharides or glycoconjugates to be used in the specificity exchangers described herein. This method utilizes glycosyltransferase, which is capable of stereoselectively forming a glycosidic linkage without any protection. In the past, this method has not reached its potential due to the fact that available glycosyltransferase is limited in kind and is expensive. In recent years, however, genes of various glycosyltransferases have been isolated and a large-scale production of glycosyltransferase by genetic techniques is common place.

U. Zehavi et al. reports a solid phase synthesis method that can be used to manufacture some of the specificity exchangers described herein, whereby a glycosyltransferase and a polyacrylamide gel bound with an aminohexyl group on a solid phase carrier is used. (See Carbohydr. Res., 124, 23 (1983), Carbohydr. Res., 228, 255 (1992), hereby expressly incorporated by reference in its entirety). This method comprises the steps of converting a suitable monosaccharide to 4-carboxy-2-nitrobenzylglycoside, condensing this glycoside with the amino group of the above-mentioned carrier, elongating the sugar chain by glycosyltransferase using the condensate as a primer, and releasing the oligosaccharide by photolysis.

In the past, there was a common understanding that glycosyltransferase does not react well with saccharide or oligosaccharide bound to a solid phase carrier, and that efficient elongation of a sugar chain is difficult to achieve. However, more recently it has been discovered that the linkage between 4-carboxy-2-nitrobenzylglycoside and solid phase carrier by a linker having a long chain, such as hexamethylene and octamethylene, improved sugar transfer yield at the maximum of 51% (React. Polym., 22, 171 (1994), Carbohydr. Res., 265, 161 (1994)).

C. H. Wong et al. report a method of enzymatic synthesis whereby glycosyltransferase is used to elongate sugar residues bound to aminated silica and, once complete, the elongated sugar chain is cleaved from the support using α-chymotrypsin. (See J. Am. Chem. Soc., 116, 1136 (1994), which is hereby expressly incorporated by reference in its entirety). By this method, the transglycosylation yield was 55%. Similarly, M. Meldal et al. reports another method of elongating a sugar chain using glycosyltransferase and a polymer of mono- and diacryloyl compound of diaminated poly(ethylene glycol) as a primer. The sugar chain was released by trifluoroacetic acid. (See J. Chem. Soc., Chem. Commun., 1849 (1994), which is hereby expressly incorporated by reference in its entirety). As mentioned above, when a sugar chain is elongated by glycosyltransferase on a solid phase carrier, the kind of group (linker) that connects the solid phase carrier to the sugar residue (receptor of initial transglycosylation) varies transglycosylation yield. When the sugar chain is liberated from the carrier, the presence of a specifically cleavable bond in the linker is desired. In sugar chain elongation by glycosyltransferase, the use of an immobilized glycosyltransferase that permits repetitive use is also desired. Preferably, if an immobilized glycosyltransferase is used for sugar chain elongation, the reaction is carried out on a water soluble carrier.

U.S. Pat. No. 6,046,040, issued to Nishiguchi et al. (2000), which is hereby expressly incorporated by reference in its entirety, describes sugar chain synthesis using an immobilized glycosyltransferase and a water soluble carrier. Accordingly, by one approach to generate the sugar-containing antigenic domains described herein, the following steps can be employed: (i) binding a sugar residue to the side chain of a water-soluble polymer via a linker having a selectively cleavable linkage to give a primer, and bringing said primer into contact with an immobilized glycosyltransferase in the presence of a sugar nucleotide, to transfer a sugar residue of said sugar nucleotide to the sugar residue of said primer, (ii) elongating a sugar chain by transfer of plural sugar residues by repeating the step (i) at least once, (iii) removing, where necessary, a by-produced nucleotide or an unreacted sugar nucleotide, and (iv) repeating the steps (i)-(iii) where necessary and releasing the sugar chain by selectively cleaving the cleavable linkage in the linker, from the above-mentioned primer connecting the sugar chain elongated by the transfer of plural sugar residues. The methods disclosed in U.S. Pat. No. 6,046,040 can be used to synthesize glycoconjugates having an optional sugar chain structure, such as oligosaccharides, glycopeptides and glycolipids, as well. The application of enzymes to an automated scheme of saccharide or glycoconjugate synthesis is also possible. Both solution and solid-phase methods can be used for automated synthesis.

In some embodiments, an apparatus that utilize enzymes to synthesize saccharides and glycoconjugates can be used herein. U.S. Pat. No. 5,583,042, which is hereby expressly incorporated by reference in its entirety, for example, describes an apparatus that utilizes combinations of glycosyltransferases, for the synthesis of specific saccharides and glycoconjugates. The next section describes several cell-based approaches to manufacture specificity exchangers comprising saccharides or glycoconjugates.

Cell Based Synthesis

In addition to using in vitro enzyme catalyzed reactions, any available cell-based methods can be used to synthesize the saccharides and glycoconjugates described herein. U.S. Pat. No. 6,458,937, which is hereby expressly incorporated by reference in its entirety, describes several cell based protocols for synthesizing saccharides and glycoconjugates. By one approach to synthesize the specificity exchangers described herein saccharides and glycoconjugates are first made by (a) contacting a cell with a first monosaccharide, and (b) incubating the cell under conditions whereby the cell (i) internalizes the first monosaccharide, (ii) biochemically processes the first monosaccharide into a second saccharide, (iii) conjugates the saccharide to a carrier to form a glycoconjugate, and (iv) extracellularly express the glycoconjugate to form an extracellular glycoconjugate comprising a selectively reactive functional group. By then reacting the glycoconjugate containing the functional group with a specificity exchanger comprising a reactive functional group, the glycoconjugate and specificity exchanger are joined. Subject compositions can include cyto-compatible monosaccharides comprising a nitrogen or ether linked functional group, for example, that are selectively reactive with similar groups present on a specificity exchanger.

By another approach, the saccharides and glycoconjugates can be synthesized by a) contacting a cell with a first monosaccharide comprising a first functional group, and b) incubating the cell under conditions whereby the cell (i) internalizes the first monosaccharide, (ii) biochemically processes the first monosaccharide into a second monosaccharide which comprises a second functional group, (iii) conjugates the second monosaccharide to a carrier to form a glycoconjugate comprising a third functional group, and (iv) extracellularly expresses the glycoconjugate to form an extracellular glycoconjugate comprising a fourth, selectively reactive, functional group.

Extracellular glycoconjugates synthesized by the above method may be presented in multiple forms such as membrane-associated (e.g., a membrane bound glycolipid or glycoprotein), associated with cell-proximate structures (e.g., extracellular matrix components or neighboring cells), or in a surrounding medium or fluid (e.g., as a secreted glycoprotein). The selective reactivity of the fourth functional group permits selective targeting of the glycoconjugate as presented by the cell. For example, fourth functional groups of surface associated glycoconjugates can provide a reactivity that permits the selective targeting of the glycoconjugate in the context of the associated region of the cell surface. Preferentially reactivity may be affected by a more reactive context. For example, the glycoconjugate-associated fourth functional group provides greater accessibility, greater frequency or enhanced reactivity as compared with such functional groups present proximate to the site of, but not associated with the glycoconjugate. In a preferred embodiment, the fourth functional group is unique to the region of glycoconjugate presentation.

The selective reactivity provided by the fourth functional group may take a variety of forms including nuclear reactivity, such as the neutron reactivity of a boron atom, and chemical reactivity, including covalent and non-covalent binding reactivity. In any event, the fourth functional group should be sufficient for the requisite selective reactivity. A wide variety of chemical reactivities may be exploited to provide selectivity, depending on the context of presentation. For example, fourth functional groups applicable to cell surface-associated glycoconjugates include covalently reactive groups not normally accessible at the cell-surface, including alkenes, alkynes, dienes, thiols, phosphines and ketones. Suitable non-covalently reactive groups include haptens, such as biotin, and antigens such as dinitrophenol.

In more embodiments, the nature of the expressed glycoconjugate is a function of the first monosaccharide, the cell type and incubation conditions. In these embodiments, the resident biochemical pathways of the cell act to biochemically process the first monosaccharide into the second monosaccharide, conjugate the second monosaccharide to an intracellular carrier, such as an oligo/polysaccharide, lipid or protein, and extracellularly express the final glycoconjugate. Alternatively, the expressed glycoconjugate may also be a function of further manipulation. For example, the fourth functional group may result from modifying the third functional group as initially expressed by the cell. For example, the third functional group may comprise a latent, masked or blocked group that requires a post-expression treatment, e.g., chemical cleavage or activation, in order to generate the fourth functional group. Such treatment may be effected by enzymes endogenous to the cell or by exogenous manipulation. Hence, the third and fourth functional groups may be the same or different, depending on cellular or extracellular processing events.

As indicated, a functional group can be a masked, latent, inchoate or nascent form of another functional group. Examples of masked or protected functional groups and their unmasked counterparts are provided in TABLE 3. Masking groups may be liberated in any convenient way; for example, ketal or enols ether may be converted to corresponding ketones by low pH facilitated hydrolysis. Alternatively, many specific enzymes are known to cleave specific protecting groups, thereby unmasking a functional group.

TABLE 3

| Masking group | Unmasked group |
|---|---|
| dialkyl ketal | ketone |
| acetal | aldehyde |
| enol ether | ketone or aldehyde |
| oxime | ketone |
| hydrazone | ketone |
| thioester | thiol |
| cobalt-complexed alkyne | alkyne |

In contrast, the nature of the intracellular glycoconjugate (comprising the third functional group) is generally solely a function of the first monosaccharide, the cell type and incubation conditions. For example, the first and second monosaccharides and the saccharide moiety incorporated into the intracellular glycoconjugate (as well as the first, second and third functional groups) may be the same or different depending on cellular processing events. For example, the first monosaccharide or functional group, cell and conditions may interact to form a chemically distinct second monosaccharide or functional group, respectively. For example, many biochemical pathways are known to interconvert monosaccharides and/or chemically transform various functional groups. Hence, predetermined interconversions are provided by a first monosaccharide, cell and incubation condition selection.

The first monosaccharide is selected to exploit permissive biochemical pathways of the cell to effect expression of the extracellular glycoconjugate. For example, many pathways of sialic acid biosynthesis are shown to be permissive to a wide variety of mannose and glucose derivatives. The first functional group may be incorporated into the first monosaccharide in a variety of ways. In preferred embodiments, the functional group is nitrogen or ether linked.

A wide variety of cells may be used according to the disclosed methods including eukaryotic, especially mammalian cells (e.g., pigs, mice, and New World monkeys) and prokaryotic cells. The cells may be in culture, e.g., immortalized or primary cultures, or in situ, e.g., resident in the organism.

The methods herein are also directed to forming products attached to the cell. Generally, these methods involve expressing an extracellular glycoconjugate as described above wherein the expressed glycoconjugate is retained proximate to the cell; for example, by being bound to membrane or extracellular matrix components. Then the fourth functional group is contacted with an agent which selectively reacts with the fourth functional group to form a product.

A wide variety of agents may be used to generate a wide variety of products. Generally, agent selection is dictated by the nature of the fourth functional group and the desired product. For example, with chemically reactive fourth functional groups, the agent provides a fifth functional group that selectively chemically reacts with the fourth functional group. For example, where the fourth functional group is a ketone, suitable fifth functional groups include hydrazines, hydroxylamines, acyl hydrazides, thiosemicarbazides and beta-aminothiols. In other embodiments, the fifth functional group is a selective noncovalent binding group, such as an antibody idiotope. In yet other embodiments, suitable agents include radioactivity such as alpha particles which selectively react with fourth functional groups comprising radiosensitizers such as boron atoms; oxidizers such as oxygen which react with fourth functional groups comprising a surface metal complex, e.g., to produce cytotoxic oxidative species; etc. Alternatively, a functional group on the cell surface might have unique properties that do not require the addition of an external agent (e.g., a heavy metal which serves as a label for detection by electron microscopy). Further examples of products formed by the interaction of a cell surface functional group and an agent are given in TABLE 4.

TABLE 4

| Functional group | Agent | Product |
|---|---|---|
| ketone | hydrazide | hydrazone |
| diene | dienophile | Diels-Alder adduct |
| thiol | alpha-bromo amide | thioether |
| boron | neutrons | radiation |
| biotin | avidin | biotin-avidin complex |
| dinitrophenol (DNP) | anti-DNP antibodies | DNP-antibody complex |
| Fluorescein | UV light | green light |
| iron complex | oxygen | peroxy radicals |

Frequently, the agent comprises an activator moiety, which provides a desired activity at the cell. A wide variety of activator moieties may be used, including moieties which alter the physiology of the cell or surrounding cells, label the cell, sensitize the cell to environmental stimuli, alter the susceptibility of the cell to pathogens or genetic transfection, etc. Exemplary activator moieties include toxins, drugs, detectable labels, genetic vectors, molecular receptors, and chelators.

A wide variety of compositions useful in the disclosed methods are provided herein. These compositions include cyto-compatible monosaccharides comprising a functional group, preferably a nitrogen or ether linked functional group, which group is selectively reactive at a cell surface. Exemplary functional groups of such compounds include alkynes, dienes, thiols, phosphines, boron and, especially, ketones. The term substituted or unsubstituted alkyl is intended to encompass alkoxy, cycloalkyl, heteroalkyl, and similar compounds. Similarly, the term substituted or unsubstituted aryl is intended to encompass aryloxy, arylalkyl (including arylalkoxy, etc.), heteroaryl, arylalkynyl, and similar compounds. The term substituted or unsubstituted alkenyl is intended to analogously encompass cycloalkenyl, heteroalkenyl, etc. Analogous derivatives are made with other monosaccharides having permissive pathways of bioincorporation. Such monosaccharides are readily identified in convenient cell and protein-based screens, such as described below. For example, functionalized monosaccharides incorporated into cell surface glycoconjugates can be detected using fluorescent labels bearing a complementary reactive functional group. A cell-based assay suitable for mechanized high-throughput optical readings involves detecting ketone-bearing monosaccharides on cell surfaces by reaction with biotin hydrazide, followed by incubation with FITC-labeled avidin and then quantitating the presence of the fluorescent marker on the cell surface by automated flow cytometry. A convenient protein-based screen involves isolating the glycoconjugate (e.g., gel blots), affinity immobilization, and detecting with the complementary reactive probe (e.g., detone-bearing glycoconjugates detected with biotin hydrazide), followed by incubation with avidin-alkaline phosphatase or avidin-horseradish peroxidase. Alternatively, monosaccharides bearing unusual functional groups can also be detected by hydrolysis of the glycoconjugate followed by automated HPLC analysis of the monosaccharides. The following section describes several approaches to manufacture the specificity exchangers described herein that utilize methods of chemical synthesis.

Chemical Synthesis

In addition to using enzyme catalyzed methods and cell-based methods, the specificity exchangers comprising saccharides and glycoproteins can be made using methods directed to chemical synthesis. Examples of methods used to synthesize saccharides and glycoconjugates can be found in Pamela Sears et al., Toward Automated Synthesis of Oligosaccharides and Glycoproteins, Carbohydrates and Glycobiology 291 Science 2344 (Mar. 23, 2001), which is hereby incorporated by reference in its entirety. Most methods of chemical synthesis involve the activation of the anomeric leaving group with a Lewis acid. The Koenigs-Knorr method of coupling glycosyl halides, one of the first techniques to gain widespread usage, is still in common use, and most other glycosidation reagents used to date proceed by the same basic mechanism.

Chemical synthesis of saccharides and glycoconjugates can also be performed automatically. Generally for automated synthesis, it is convenient for the reactions to be performed on solid phase. This approach allows the rapid removal of reactants, relatively easy purifications, and (in the case of library construction) the encoding of the product either by position (as in a two-dimensional array “chip” format) or, for “mix and split” type library construction, by an accessory encoding reaction, in which the labels are added to the solid support as the chain is extended or by radio frequency-encoded combinatorial chemistry technology. Hydrophilic supports, such as polyethylene glycol-based resins, have been used with good success, as have “hybrid” resins, such as Tentagel, that have a polystyrene core coated in polyethylene. To a lesser extent, soluble supports, such as polyethylene glycols and derivatives, have been used in saccharide synthesis.

Another approach that can be used for saccharide and glycoconjugate synthesis is a one-pot reaction. One-pot reactions rely on the reactivity profile of different protected sugars to determine the synthesized product. The reactivity of a sugar is highly dependent on the protecting groups and the anomeric activating group used. By adding substrates in sequence from the most reactive to least reactive, one can assure the predominance of a desired target compound. The key to this approach is to have extensive quantitative data regarding the relative reactivities of different protected sugars, which is currently being generated by those with skill in the art of glycomics. These reactions are typically performed in solution, but in order to facilitate removal of reactants at the end, the final acceptor may be attached to a solid phase.

This approach can be made even more efficient through automation, such as a computer program. Compared with stepwise solid-phase synthesis, the one-pot approach uses protecting-group manipulation only at the stage of building block synthesis and thus holds greater potential for automation and for greater diversity of oligosaccharide structures.

Additionally, several other methodologies can be employed to synthesize the glycopeptides and glycoproteins that are joined to or incorporated in the specificity exchangers described herein. Several of these methods are discussed in Pamela Sears et al., Toward Automated Synthesis of Oligosaccharides and Glycoproteins, Carbohydrates and Glycobiology 291 Science 2344 (Mar. 23, 2001), which is hereby incorporated by reference in its entirety. By one approach, for example, attachment of saccharide chains to the specificity exchangers described herein is accomplished in a stepwise fashion, beginning from the nonreducing end and proceeding to the reducing end. As is the case with glycal-based synthetic schemes and the one-pot strategy outlined above, the ultimate acceptor can be an amino acid, peptide or glycopeptide. For coupling to hydroxylated amino acids, such as serine or threonine, the chemistry is very much the same as that used to construct the glycosidic bonds: the activated anomeric position is directly attacked by a deprotected hydroxyl group on the peptide. In the case of NH₂-linked glycosides, the reducing-end sugar is typically prepared first as a sugar azide, which is then reduced and coupled to a free aspartate via carbodiimide activation. The acceptor can be an amino acid, for which the product can be incorporated into solid-phase peptide synthesis (SPPS) schemes to produce the target glycopeptide, or it may itself be the final polypeptide. Glycosylated amino acids bearing typically one to three sugars have been used successfully in solid phase synthesis of many glycopeptides.

In certain embodiments the glycopeptide containing specificity exchangers described herein can be synthesized by glycosylating the peptide in a stepwise fashion from the reducing to the nonreducing end through chemical or enzymatic methods. Typically, a single glycosylated peptide is made by SSPS, the sugar is selectively deprotected, and the oligosaccharide is built up in a stepwise fashion. The singly glycosylated peptide can be constructed via SPPS, and the sugar can be completely deprotected to provide the substrate for the action of three successive glycosyltransferases. The synthesis of these glycopeptides can also be automated.

Extension of glycosylated peptides into glycoproteins can also be accomplished by a number of approaches. Workers (Allen, P. Z., and Goldstein, I. J, Biochemistry, 1967, 6, 3029; Rude, E., and Delius, M. M., Carbohydr. Res., 1968, 8, 219; Himmelspach, K., et al., Eur. J. Immunol, 1971, 1, 106; Fielder, R. J., et al, J. Immunol., 1970, 105, 265) developed several techniques for conjugation of carbohydrates to protein carriers, for example. Most of them suffered by introducing an antigenic determinant in the linker itself, resulting in generation of polyclonal antibodies. Kabat (Arakatsu, Y., et al., J. Immunol., 1966, 97, 858), and Gray (Gray, G. R., Arch. Biochem. Biophys. 1974, 163, 426) developed conjugation methods that relied on oxidative or reductive coupling, respectively, of free reducing oligosaccharides. The main disadvantage of these techniques, however, is that the integrity of the reducing end of the oligosaccharide was compromised. In 1975 Lemieux described the use an 8-carbomethoxy-1-octanol linker (Lemieux, R. U., et al., J. Am. Chem. Soc., 1975, 97, 4076) which alleviated the problem of linker antigenicity and left the entire oligosaccharide intact. Equally effective in producing glycoconjugates was the allyl glycoside method described by Bernstein and Hall. (Bernstein, M. A., and Hall, L. D., Carbohydr, Res., 1980, 78, C1.) In this technique the allyl glycoside of the deblocked sugar is ozonized followed by a reductive workup. The resultant aldehyde is then reductively coupled to a protein carrier with sodium cyanoborohydride.

Short peptides can also be coupled to larger ones by “native peptide ligation” strategies. Easier approaches to glycoprotein synthesis can be achieved through cell based methods, however. The glycans produced by this method will be determined by many factors, including the local protein structure around the glycosylation site and the relative amounts of glyco-processing enzymes produced in the cell. Many of these factors also vary with the cell line, so a glycoprotein produced in one cell line may have different glycosylation than the same protein produced in another cell line.

The resulting products however can be used as a starting point for many schemes in which the sugar chain is digested down to a simple homogeneous core and then reelaborated enzymatically. For example, N-glycosylated proteins can have the glycans digested down to the innermost N-acetylglucosamine by using endoglycosidases, thus converting a heterogeneous population to a homogeneous one in which each glycosylation site has only a single sugar attached. These simple glycoproteins can then be elaborated enzymatically to increase the size and complexity of the glycan by using glycosyltransferases or endoglycosidase-catalyzed transglycosylation. The transglycosidase approach is limited by the substrate specificity of the endoglycosidases, which are enzymes that cleave between the innermost N-acetylglucosamine residues of N₂-linked oligosaccharides. In certain embodiments the endoglycosidase can be endoglycosidase M from Mucor hiemalis, which accepts a wide range of high-mannose-, hybrid- and complex-type glycans.

Another option is to remove the glycosylated sections by using proteases and then reattach short, chemically synthesized glycopeptides in their place. This ligation can be accomplished enzymatically through the use of proteases or inteins, self-splicing polypeptides that are able to excise themselves from proteins posttranslationally. In the latter case, the peptide segment to be replaced is substituted at the genetic level with the sequence encoding the intein.

Proteases can catalyze peptide synthesis using either the thermodynamic approach or the kinetic approach. In the thermodynamic approach, peptides are condensed to form the larger product typically by precipitation of the product or by conducting the reaction in a solvent with low water activity. A more useful approach, as far as enzyme activity, stability, and solubility are concerned, is the kinetic approach, in which a peptide ester undergoes a competition between hydrolysis and aminolysis. The ratio of aminolysis to hydrolysis can be improved by adding an organic cosolvent to lower the water concentration and suppress amine ionization, by increasing the amine nucleophile concentration, or by modifying the enzyme active site. With regard to enzyme modification, the conversion of the active-site serine of serine proteases to a cysteine has been shown to be highly effective for creating a peptide ligase. Glycosylation of proteins has long been known to render them less susceptible to protease activity, and so it might be inferred that glycopeptides would be difficult to couple using proteases. A systematic study of subtilisin-catalyzed synthesis of glycopeptides, however, reveals that the protease could couple glycopeptides successfully, provided that the glycosylation site was not at the forming bond and that the coupling yields improved as the glycosylation site was placed farther away from it. One of the most effective and practical glycopeptide ester leaving groups is the benzyl-type ester generated from a modified Rink amide resin and cleaved with trifluoroacetic acid.

An alternate approach is to use intein-mediated coupling of glycopeptides to larger proteins. It is possible to intervene in the natural splicing reaction by removing the COOH-terminal extein, then allowing the reaction to be completed with an exogenously added nucleophile, which may be a glycopeptide. As in the native peptide ligation strategy, the peptide preferably contains a cysteine at the NH₂-terminus.

Glycoprotein purification procedures can be very similar to the purification of unglycosylated proteins. The first step in glycoprotein purification is usually to solublize the glycoprotein. Glycoproteins that are secreted into the media are relatively easy to purify if serum free media has been used to grow the cells. Glycoproteins that remain trapped in a vesicle (as seen with chicken Thy-1) can be solublized with detergents. Once in detergent, the proteins can be dialyzed against aqueous buffers.

After solublizing the glycoprotein, various chromatographic purification schemes can be used to purify it. In certain embodiments, Lectin Affinity Chromatography can be used. Lectins are non-immune proteins or glycoproteins that bind to specific saccharides and glycoconjugates with high affinity. Because of their binding specificity, lectins show a range of specificities for carbohydrates and glycoconjugates. These lectins can easily be immobilized onto a variety of supports and used for affinity chromatography. Once coupled, lectins are stable with most of the buffers.

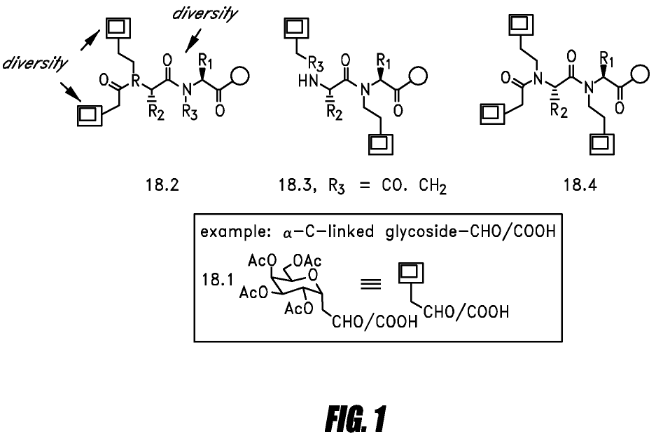

Research carried out by Arya, et al., has lead to development of an automated, multi-step, solid-phase strategy for the parallel synthesis of artificial glycopeptide libraries. (Arya, P. et. al., 7 Med. Chem. Lett. 1537, 1997, herein expressly incorporated by reference in its entirety). In some embodiments, the specificity exchangers described herein are constructed using this strategy. FIG. 1 illustrates this approach.

Accordingly, different α- or β-carbon linked carbohydrate based aldehyde and carboxylic acid derivatives, protected as acetates (see 18.1 in FIG. 1) can be incorporated either at the N-terminal moiety or at the internal amide nitrogen of short peptides/pseudopeptides (e.g., specificity domains or specificity domains joined to an antigenic domain) in a highly flexible and controlled manner. The chain length of the C-glycoside can be varied and the carbohydrate moiety can be synthesized in either the pyranose or furanose form. Monosaccharides and their derivatives are not the only available carbohydrate building blocks. In certain embodiments, disaccharides and higher order oligosacccharides can also be used as carbohydrate building blocks. C-Glycosides are generally more stable to enzymatic and acid/base hydrolysis than their oxygen counterparts. This method is more versatile than the glycosylated amino acid building block in which the choice of amino acids is limited.

Using this approach, libraries of artificial glycopeptides can be readily synthesized for probing carbohydrate-protein interactions. Several “working models” that display multiple copies of carbohydrates have been developed (see 18.2, 18.3, and 18.4 in FIG. 1) while the dipeptide scaffold may contribute to secondary interactions with the biological target. (Arya, P. et al., 8 Med. Chem. Lett. 1127, 1998; Arya, P. et al., 7 Med. Chem. 2823, 1999).

Initially, artificial glycopeptides were synthesized by a convergent strategy on a peptide synthesizer. (Kutterer, et. al., 1 J. Comb. Chem. 28, 1999). The synthesis of these artificial glycopeptide libraries has been successfully transferred to a fully automated multiple organic synthesizer and each step in the synthesis was optimized. (Arya, P. et al., 2 Comb. Chem. 120, 2000). This methodology involves coupling an amino acid to a solid-support resin such as Rink amide MBHA resin or TentaGel derivatized Rink amide resin. After removal of the protecting group on the amino acid, the sugar aldehyde undergoes reductive amination (see 18.3 and 18.4 in FIG. 1) with the resin bound amino group followed by amino acid coupling of the second amino acid. After deprotection of the amino acid, a second reductive amination can occur and/or a sugar acid can be coupled. The sugar moieties are then deacetylated, and the compounds are cleaved from the resin. The synthesis of a 96 compound library can be obtained from just 24 dipeptides and two sugar aldehydes. (See Randell, Karla D., et al., High-throughput Chemistry toward Complex Carbohydrates and Carbohydrate-like Compounds, National Research Council of Canada, publication no. 43876, Feb. 13, 2001).

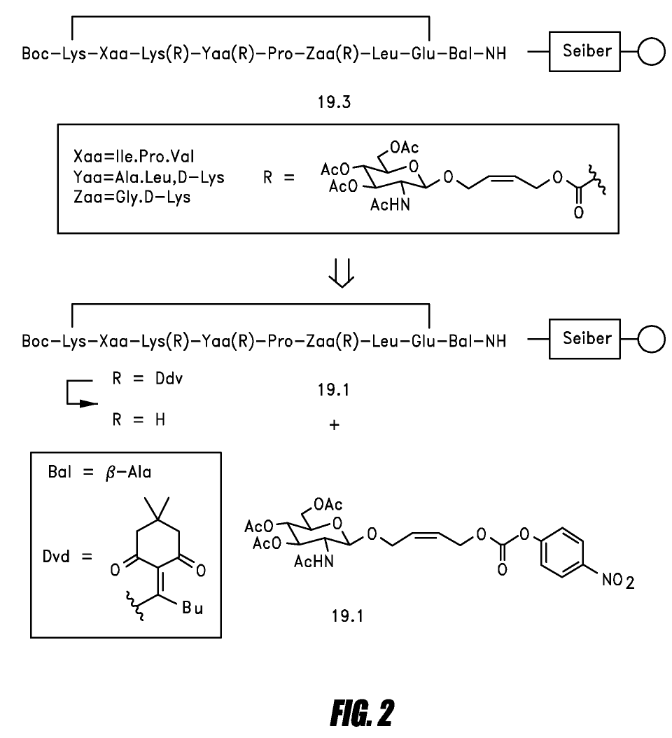

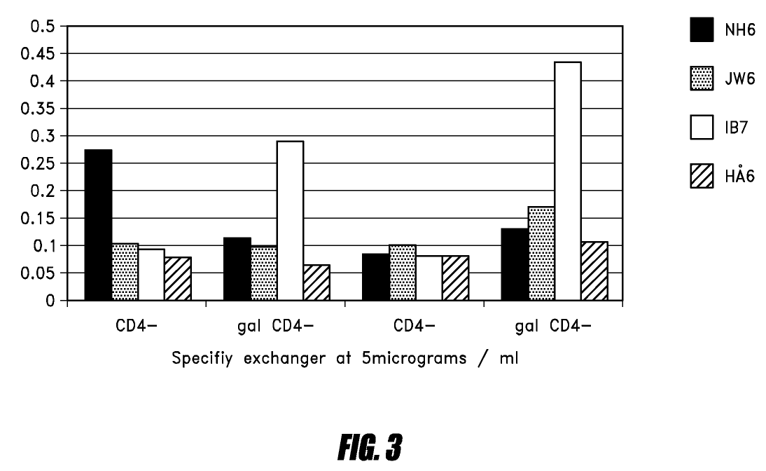

A recent article describes another approach that can be used to manufacture the specificity exchangers described herein. The synthesis of multivalent cyclic neoglycopeptides has been accomplished. (See Wittmann, V.; Seeberger, et al., 39 Chem. Int, Ed. 4348, 2000, herein expressly incorporated by reference in its entirety). A new urethane-type linker based on the Alloc protecting group was developed for the glycosylation reaction, which proceeds virtually quantitatively. A library of cyclic peptides (e.g., specificity exchangers) can be synthesized using the split and mix method on TentaGel resin linked via the Sieber linker. FIG. 2 illustrates this approach.

The synthesis reaction shown in FIG. 2 can be monitored by withdrawal of a small amount of resin from the well and analysis by HPLC in combination with electrospray mass spectrometry. The p-nitrophenyl carbonate derivative of the sugar moiety (see 19.2 in FIG. 2) was attached to three points of the cyclic peptide in one step using a five-fold excess (per attachment point) of the sugar in the presence of DIPEA. A library of eighteen cyclic neoglycopeptides (see 19.3 in FIG. 2) can be efficiently synthesized. This methodology can be applied to the synthesis of many different libraries by varying the distances between the carbohydrate moieties as well as the carbohydrate moiety itself. The section below discusses the incorporation of linkers to the specificity exchangers and/or saccharides or glycoconjugates.

Linkers

In certain embodiments, the saccharide or glycoconjugates can be joined to the specificity exchangers through linkers or by association with a common carrier molecule, as discussed previously. In some embodiments linkers are used to join saccharides to at least one amino acid of the specificity exchangers. In general the term “linkers” refers to elements that promote flexibility of the molecule, reduce steric hindrance, or allow the specificity exchanger to be attached to a support or other molecule. Any suitable linker can be used to attach the saccharide and or glycoconjugate to a specificity exchanger. In certain embodiments the linker can be polyethylene glycol.

Other types of linkers that can be incorporated with a specificity exchanger include avidin or streptavidin (or their ligand—biotin). Through a biotin-avidin/streptavidin linkage, multiple specificity exchangers can be joined together (e.g., through a support, such as a resin, or directly) or individual specificity domains can be joined to a saccharide or glycoconjugate.

Another example of a linker that can be included in a specificity exchanger is referred to as a “λ linker” because it has a sequence that is found on λ phage. Preferred λ sequences are those that correspond to the flexible arms of the phage. These sequences can be included in a specificity exchanger (e.g., between the specificity domain and the saccharide or glycoconjugate or between multimers of the specificity and/or saccharides and glycoconjugates) so as to provide greater flexibility and reduce steric hindrance.

The Example below describes an approach that was used to synthesize several glycosylated ligand/receptor specificity exchangers.

### EXAMPLE 1

Several glycosylated ligand/receptor specificity exchangers (see TABLE 1) comprising a specificity domain corresponding to a CD4 receptor region that interacts with the HIV-1 glycoprotein 120 (see Mizukami T., Fuerst T. R., Berger E. A. & Moss B., Proc Natl Acad Sci USA. 85, 9273-7 (1988), herein expressly incorporated by reference in its entirety, were synthesized on solid phase. The peptides were produced in an automatic synthesis robot using Fmoc chemistry (see Ed. Chan W. C. & White P. D, Fmoc solid phase peptide synthesis-a practical approach (2000) Oxford university press., herein expressly incorporated by reference in its entirety). Each peptide, still attached to the solid support (resin), was divided in a minor and a major fraction. The minor peptide fractions were cleaved off the resin by treatment with TFA, while the major fractions were left attached to the resin, awaiting glycosylation. The cleaved peptides were analysed by reversed phase HPLC (λ=220 nm) to check the purity. After analysis, the cleaved peptides were lyophilised.

A reagent including the sugar Gal-α1-3Gal has been shown to absorb human anti-Gal-α 1-3Gal antibodies (Rieben R., von Allmen E., Korchagina E. Yu. Et al. Xenotransplantation. 2, 98-106 (1995), herein expressly incorporated by reference in its entirety). The formula for Gal-α1-3Gal-β is provided below as Formula 1.

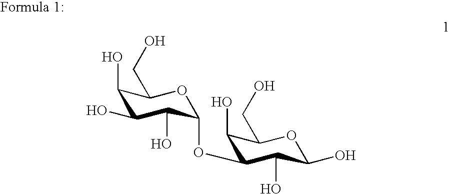

The sugar reagent (Galα1-3Galβ1-O(CH₂)₃NH₂ (Lectinity corp., product no. 88)) was coupled to the side chain of an aspartic acid (Formula 2):

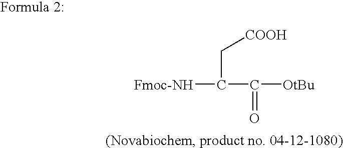

The amino acid was protected both at the N-terminal and C-terminal ends. A covalent bond between the sugar amino group and the amino acid carboxyl group, was formed. (See Synthesis 1).

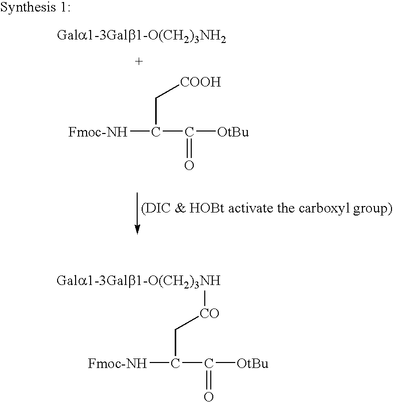

The coupling reaction was monitored by analysing samples from the reaction mixture by reversed phase HPLC using λ=266 nm since the Fmoc protecting group absorbs UV light strongly at this wavelength. The coupling reaction was stopped after ˜4-6 h. The glyco-amino acid was purified by reversed phase HPLC (λ=266 nm). The purified glyco-amino acid was lyophilised.

Next, deprotection of the glyco-amino acid was performed. To allow coupling of the glyco-amino acid to the CD4 peptides, the OtBu-protecting group was cleaved off the glyco-amino acid C-terminus, by treatment with TFA (see Synthesis 2).

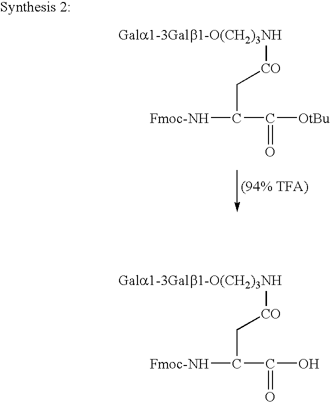

The deprotection was monitored by reversed phase HPLC (λ=266 nm). After ˜2 h, the deprotection was stopped. Most of the TFA was evaporated by nitrogen gas. The remaining solution was diluted 1/10 with water. The deprotected glyco-amino acid was purified by reversed phase HPLC (λ=266 nm). The purified and deprotected glyco-amino acid was lyophilised.

Following deprotection of the glyco-amino acid, coupling to the CD4 and CDR specificity domain peptides was performed. The glyco-amino acid was covalently linked to the N-terminal ends of the resin-bound specificity domains using Fmoc chemistry (see Synthesis 3). The coupling reaction was stopped after ˜6 h.

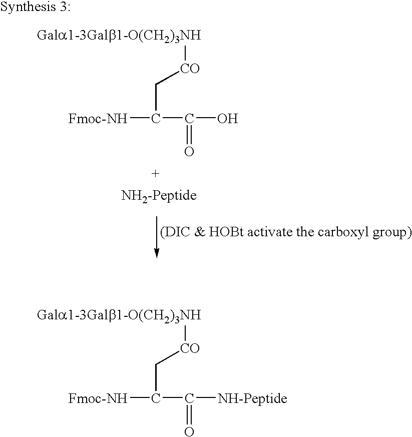

The glycosylated specificity exchangers were deprotected and cleaved off their solid supports by treatment with TFA. The cleaved glycosylated specificity exchangers were analysed by reversed phase HPLC (λ=220 nm) to check the purity in comparison with the corresponding non-glycosylated peptides. Glycosylation-specific peaks were purified by reversed phase HPLC (λ=220 nm). The purified glycosylated specificity exchangers were lyophilised. After lyophilization, a fraction of each glycosylated specificity exchanger was analysed by MALDI-MS to verify its identity.

The next Example describes a pathogen-based characterization assay that was performed to evaluate the ability of glycosylated ligand/receptor specificity exchangers to interact with HIV.

### EXAMPLE 2

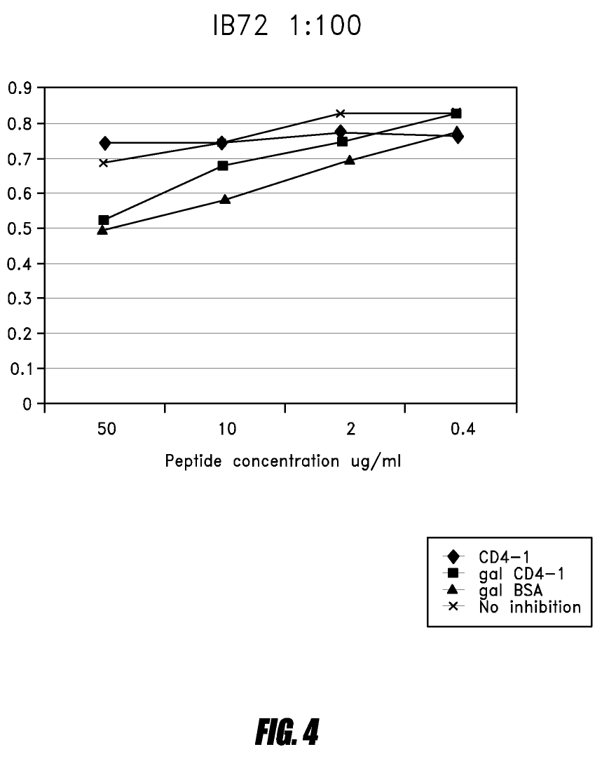

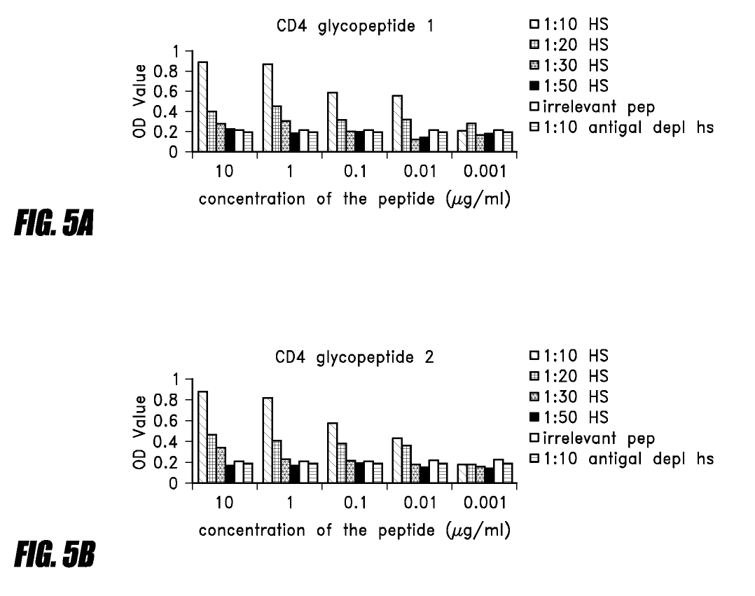

Glycosylated ligand/receptor specificity exchangers specific for HIV were produced according to the approaches described in Example 1. To evaluate the ability of glycosylated specificity exchangers to bind human Gal-alpha1,3-Gal-specific antibodies, glycosylated and non-glycosylated versions of the same ligand/receptor specificity exchanger were coated on solid phase of microtitre plates. Four human sera were allowed to bind to the coated peptides, and an enzyme labeled anti-human antibody indicated bound human antibodies. The results showed that only the glycosylated peptides were able to bind human antibodies (human sera IB72; FIG. 3).

To further evaluate the ability of the glycosylated specificity exchangers to bind to a pathogen, a glycosylated peptide competitive assay was performed using the most reactive human sera (IB72). In brief, Gal-alpha-1,3-Gal-labeled bovine serum albumin (Gal-BSA) was coated onto 96-well microplates in sodium carbonate buffer (pH: 9.6) at +4° C. overnight. Dilutions of human sera and dilutions of glyco-peptides (glycosylated HIV-specific ligand/receptor specificity exchangers or Gal-BSA) or non-glycosylated peptides (HIV-specific ligand/receptor specificity exchangers or BSA) were preincubated in phosphate-buffered saline (PBS) containing 1% bovine albumin, 2% goat serum and 0.05% Tween 20 at 37° C. for 1 h. The mixture was then added to the coated plates and incubated at 37° C. for 1 h, then washed 3 times with phosphate-buffered saline (PBS) containing 0.05% Tween 20 (pH: 7.4).

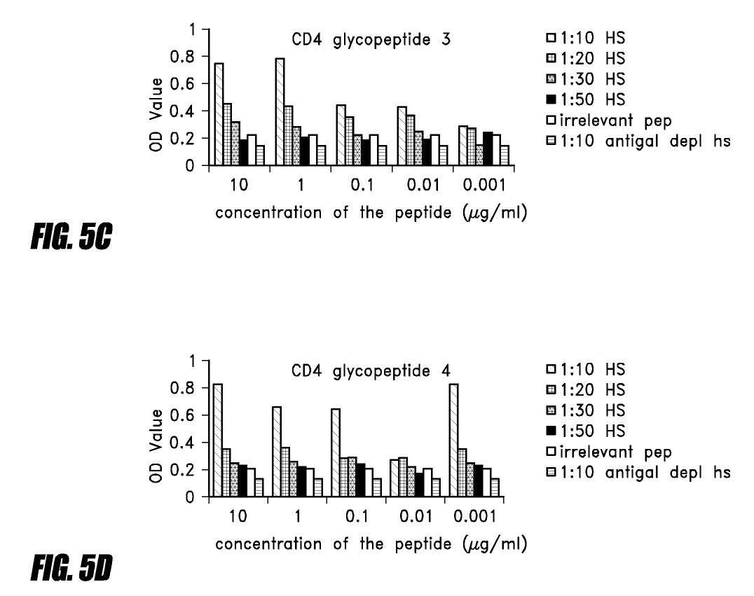

Bound antibodies were indicated with goat anti-human polyvalent Ig, conjugated with alkaline phosphatase. The plates were incubated and washed as described above. Plates were developed with phosphatase substrate at room temperature for 30 min, stopped with 1 M NaOH. Optical density (OD) at 405 nm/650 nm was determined to quantify the inhibition. The results are provided in FIG. 4, which shows that the human antibody binding to Gal-BSA could only be inhibited by either Gal-BSA or the glycosylated peptide. Thus, specificity exchangers that bind Gal-alpha1,3-Gal-specific antibodies had been generated. Gal-BSA mixed with human sera in the same conditions as mentioned above was used as positive control and 100% inhibition was observed.

The next example describes the preparation and analysis of particular glycosylated specificity exchangers that are specific for HIV.

### EXAMPLE 3

This example describes the preparation and characterization of several glycosylated specificity exchangers that can be used inhibit replication of HIV. A total of six glycosylated specificity exchangers comprising Gal-α1-3Gal-β (each 15 amino acids in length), specific for HIV were prepared, as described in EXAMPLE 1. (See TABLE 5). These glycosylated specificity exchangers comprise a specificity domain that corresponds to a CD4 receptor region that interacts with the HIV-1 gp120 coupled to Gal-α1-3Gal-β.

TABLE 5

Glycosylated Specificity Exchangers specific
for HIV

| Glycosylated | | |
|---|---|---|
| Sp.exchanger | Sequence | Identifier |
| Gal-CD4-100 | QFHWKNSNQIKILGN | (Seq. Id. No.4) |
| Gal-CD4-101 | NSNQIKILGNQGSFL | (Seq. Id. No.5) |
| Gal-CD4-102 | KILGNQGSFLTKGPS | (Seq. Id. No.6) |
| Gal-CD4-103 | QGSFLTKGPSKLNDR | (Seq. Id. No.7) |
| Gal-CD4-104 | TKGPSKLNDRADSRR | (Seq. Id. No.8) |
| Gal-CD4-105 | KLNDRADSRRSLWDQ | (Seq. Id. No.9) |

The HIV-specific glycosylated specificity exchangers (Seq. Id. Nos. 4-9) were then analyzed for their ability to bind to gp120 and for their ability to reduce or neutralize HIV infection or replication of the virus. Binding of the glycosylated specificity exchangers to gp120 was assessed using ELISA and various concentrations of the glycosylated specificity exchangers (Seq. Id. Nos. 4-9) in the presence of various dilutions of human serum. Briefly, binding of the peptides to the gp120 molecules and binding of the antibodies to the disaccharide was tested by enzyme immunoassay (EIA). 96-well microtiter plates (NUNC, Polysorp, Denmark) were coated with LAI gp120 molecule (PerkinElmer, Denmark) at 1 μg/ml in 50 mM carbonate-bicarbonate buffer and incubated 1 h at 37° C. Plates were then blocked with a 2% solution of human serum albumin (Calbiochem) in PBS-A and incubated for 2 h at 37° C. After washing, the glycosylated peptides were added at different concentrations and incubated for 1 h at 37° C. Then, AB type human serum from healthy individuals (Cambrex, USA) was added at different dilutions and incubated for 1 h. A polyvalent anti-human antibody HRP conjugated (Dako, Denmark) was added and incubated 1 h at 37° C. OPD was used as a substrate and plates were read at 490 nm after 30 min of incubation at room temperature in the dark.

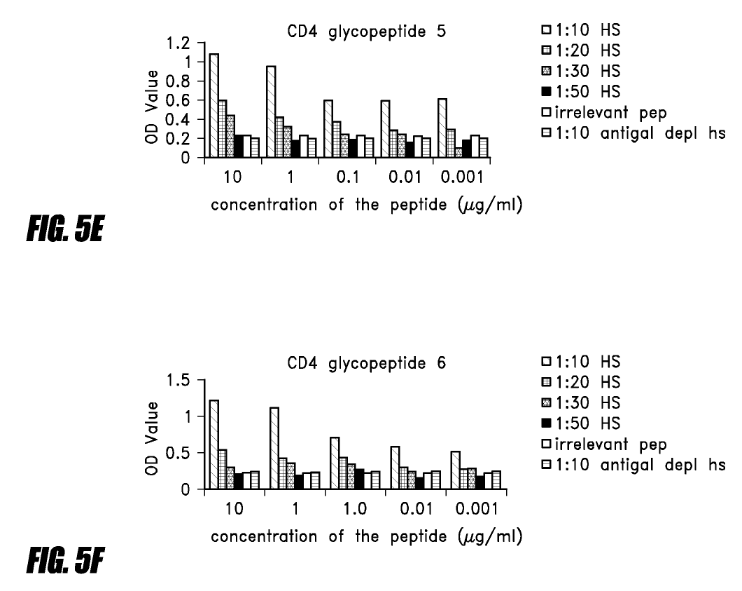

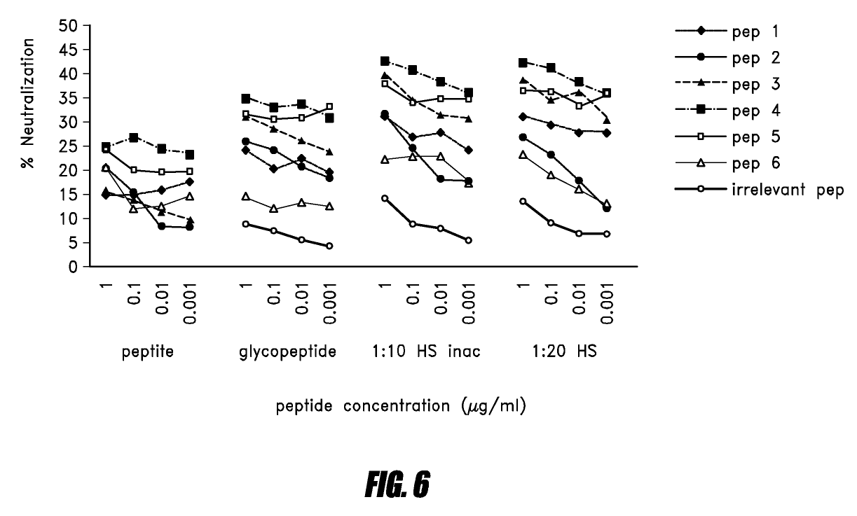

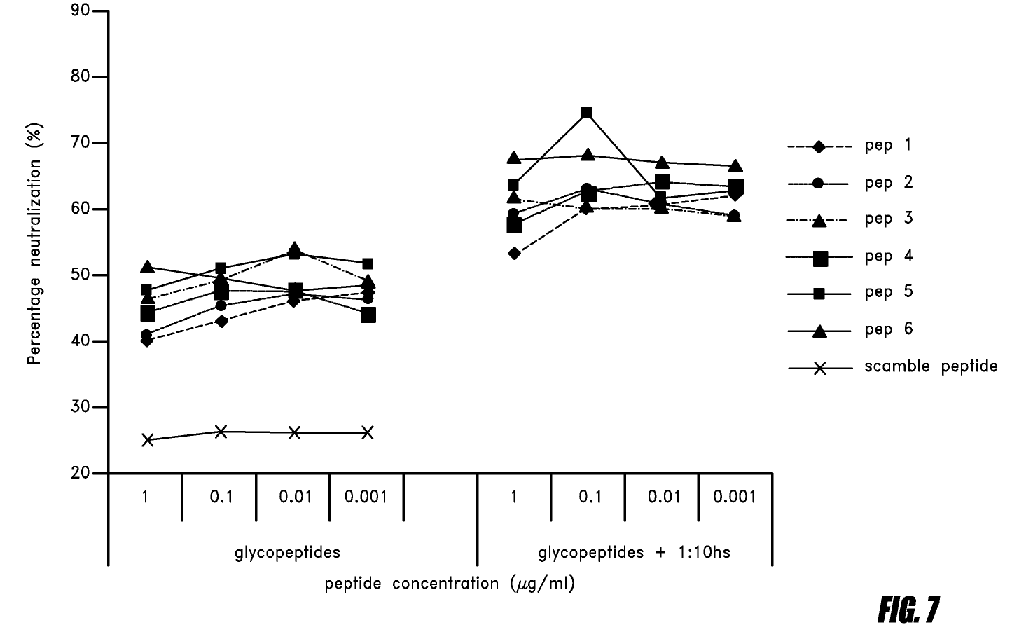

Binding of anti-gal(α1,3)gal antibodies to solid-phase bound gp120 was detected for all peptides down to a concentration of 10 ng/ml (approximately 5 nM) using a 1:10 or 1:20 dilution of human serum (FIG. 5). All peptides bound with similar effectivity to gp120 (FIG. 5). Human serum depleted of the anti-gal(α1,3)gal antibodies did not detect bound glycopeptide, confirming that antibodies bound to the plates were directed against the gal α (1,3) gal β disaccharide.

The next example describes the antiviral activity of the glycosylated specificity exchangers using a neutralization assay.

### EXAMPLE 4

Once it had been determined that the glycosylated specificity exchangers appreciably bound gp120 and human antibody, the antiviral activity of the glycosylated specificity exchangers was evaluated using a neutralization assay that is recognized in the art to reasonably predict the therapeutic efficacy of an HIV vaccine. (See Y. Shi et al., “A new cell line-based neutralization assay for primary HIV type 1 isolates,” AIDS Res Hum Retroviruses 18:957-967 (2002), herein expressly incorporated by reference in its entirety). By this approach, HIV infection of U87 cells that express CD4 and one of the major co-receptors (either CXCR4 or CCR5) were challenged with the glycosylated specificity exchangers and human serum during competition with the CD4 receptor. The infected cells form syncytia, that is, plaques, that were stained and enumerated by light microscopy. Neutralization was then determined by the ability of human serum and glycosylated specificity exchanger to reduce the number of plaque-forming units (PFU) relative to controls.

In brief, the U87 cells were seeded onto a 48 well plate at a concentration of 100,000 cell/well 1 day prior infection with HIV-1 IIIB. On the day of infection, glycosylated specificity exchangers (Seq. ID. Nos. 4-9), human serum and virus stock were diluted in culture medium and incubated in a separate 48 well plate, to a final dilution of 1:10/1:20 for the serum and an appropriate concentration of the virus, according to the titration. The glycosylated specificity exchangers were analyzed at different concentrations to determine the minimal amount required for antiviral activity. The glycosylated specificity exchanger-serum-virus mixture was kept at 37° C. for 1 hr. Then, two hundred microliters of each dilution was distributed into wells containing the U87/CD4 cells. The experiment was performed in quadruplicate in the same plate and in five separate plates.

Controls for the assay included glycosylated specificity exchanger without the coupled sugar, the glycosylated specificity exchanger (coupled with the sugar) and a 1:10/1:20 dilution of heat inactivated human serum, cells and medium only, and cells and medium and serum. Positive controls included culturing the cells with virus and no serum and infecting the cells with a mixture of the virus followed by incubation with anti-HIV monoclonal antibodies (IgG1 b12, 2F5, 2 g12), which have been shown to synergistically inhibit replication of the virus in previous studies.

The experiment was terminated at day 3 or 4 by fixation with methanol-acetone (1:1) and the number of plaque-forming units was determined after haematoxylin staining, as described by Shi et al. The results of the neutralization study are shown in TABLE 6, wherein the values are shown as the percentage of neutralization of the virus (e.g., 0.3 equals 30% inhibition of HIV replication (syncytia formation). The data revealed that the glycosylated specificity exchangers (Seq. Id. Nos. 4-9) effectively inhibited HIV replication in the presence of human serum providing strong evidence that glycosylated specificity exchangers specific for HIV can be incorporated into pharmaceuticals and medicaments that can be used to inhibit HIV replication in an infected subject and thereby treat or prevent HIV infection in said subject. The next example provides evidence that the CD4 glycoconjugated peptides described herein effectively neutralize HIV infection and induce a potent ADCC response.

TABLE 6
Neutralization Assay Using Glycosylated Specificity Exchangers

| | | | | | | | | Glycosylated | | | | Glycosylated | | | |
|---|---|---|---|---|---|---|---|---|---|---|---|---|---|---|---|
| | CD4 peptide | | | Glycosylated | | | | Sp. Exchanger &1:10 | | | | Sp. Exchanger &1:20 | | | |
| | Alone | | | Sp. Exchanger | | | | dilution human serum | | | | dilution human serum | | | |
| Seq. Id. | 10 | 1 | 0.1 | 100 | 10 | 1 | 0.1 | 100 | 10 | 1 | 0.1 | 100 | 10 | 1 | 0.1 |
| No. | ug/ml | ug/ml | ug/ml | ug/ml | ug/ml | ug/ml | ug/ml | ug/ml | ug/ml | ug/ml | ug/ml | ug/ml | ug/ml | ug/ml | ug/ml |
| 4 | neg | neg | neg | ND | neg | neg | neg | ND | 0.55 | 0.48 | 0.48 | ND | 0.48 | 0.47 | 0.47 |
| 5 | neg | neg | neg | ND | neg | neg | neg | ND | 0.49 | 0.39 | neg | ND | 0.429 | 0.32 | neg |
| 6 | neg | neg | neg | ND | ND | ND | neg | ND | 0.60 | 0.42 | 0.50 | ND | neg | neg | neg |
| 7 | neg | neg | neg | ND | neg | neg | neg | ND | 0.454 | 0.461 | 0.439 | ND | neg | neg | neg |
| 8 | ND | ND | ND | ? | 0.552 | 0.548 | ND | ND | 0.83 | 0.821 | 0.778 | ND | 0.642 | 0.667 | 0.701 |
| 9 | ND | ND | ND | neg | neg | neg | neg | 0.75 | 0.78 | 0.75 | 0.76 | 0.734 | 0.74 | 0.75 | 0.71 |
| 7 | ND | ND | ND | neg | neg | neg | neg | ND | 0.623 | 0.616 | 0.616 | ND | 0.79 | 0.775 | 0.784 |
| x2lys | | | | | | | | | | | | | | | |
| 7 | ND | ND | ND | ND | 0.32 | ND | ND | ND | 0.426 | 0.483 | 0.391 | ND | ? | ? | ? |
| x4lys | | | | | | | | | | | | | | | |
| Mixture | ND | ND | ND | ND | neg | neg | neg | ND | 0.913 | 0.935 | 0.92 | ND | 0.935 | 0.935 | 0.927 |
| of 4, 5, | | | | | | | | | | | | | | | |
| 6, and 7 | | | | | | | | | | | | | | | |

Neg = negative <0.30
ND = not determined
? = not conclusive

### EXAMPLE 5

The great variability and high glycosylation of gp120 limits the neutralization of the HIV infection. The binding region of the CD4 receptor on gp120, however, is highly conserved and therefore represents a good target. Here we describe a method by which peptides (total number of 6) corresponding to residues 25 to 64 of the CD4 receptor have been coupled to a major antigen, the gal-alpha 1,3-gal disaccharide, towards which humans have natural antibodies. It is contemplated that these CD4 glycoconjugate peptides can redirect the specificity of the antibodies towards gp120 and thereby reduce the HIV viral loads of infected individuals.

A total of six peptides, 15 amino acids each that overlap by 70% were synthesized and coupled to the gal alpha 1,3 gal disaccharide (Seq. Id. Nos. 4-9, respectively). The binding to gp120 of the glycopeptide-anti-gal α (1,3) gal β antibody complexes was examined by ELISA and their neutralization capacity was assessed in three different assays: 1) plaque assay in U87 cells 2) single round infectivity assay in TZMbl cells and 3) infectivity assay in H9 cells. In order to analyze the cytotoxic effect induced by the anti-gal antibody, the complement activity was tested by using non-heat inactivated human serum in the same setting of the neutralization assays and the antibody dependent cellular cytotoxicity (ADCC) with NK cells was studied by flow cytometry.

As described above, a standard Elisa assay was performed to confirm binding of the molecule for both, peptide to gp120 and human anti-gal antibody to the sugar residue. Several dilutions of the derived peptide and the human serum were evaluated so as to determine favorable ratios and concentrations (FIGS. 5 (A-F)).

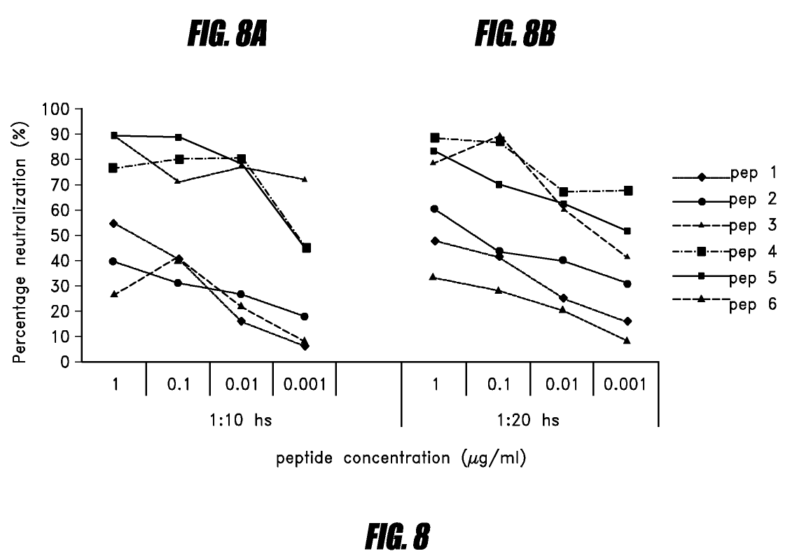

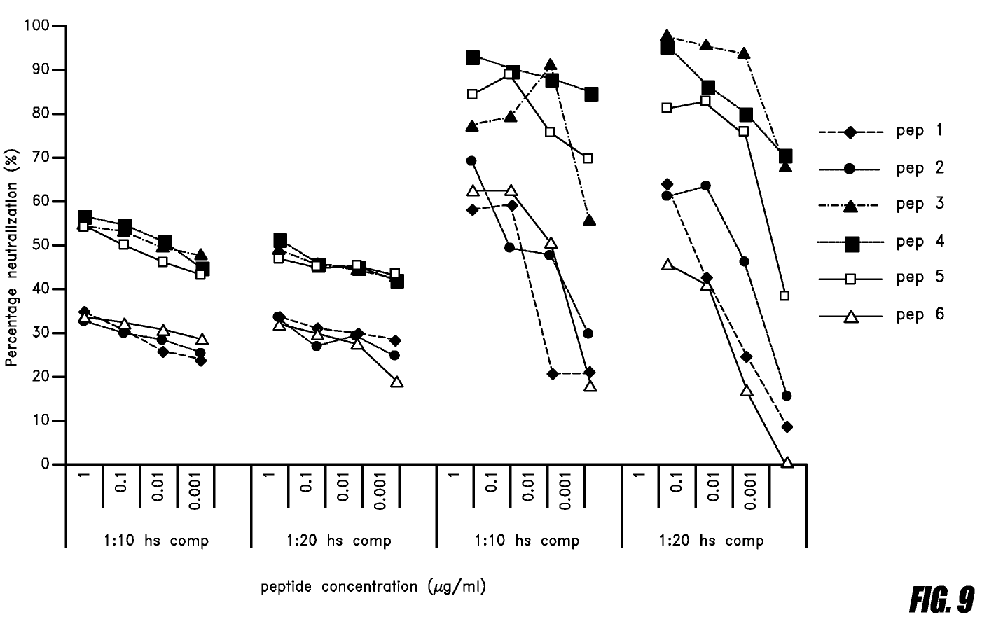

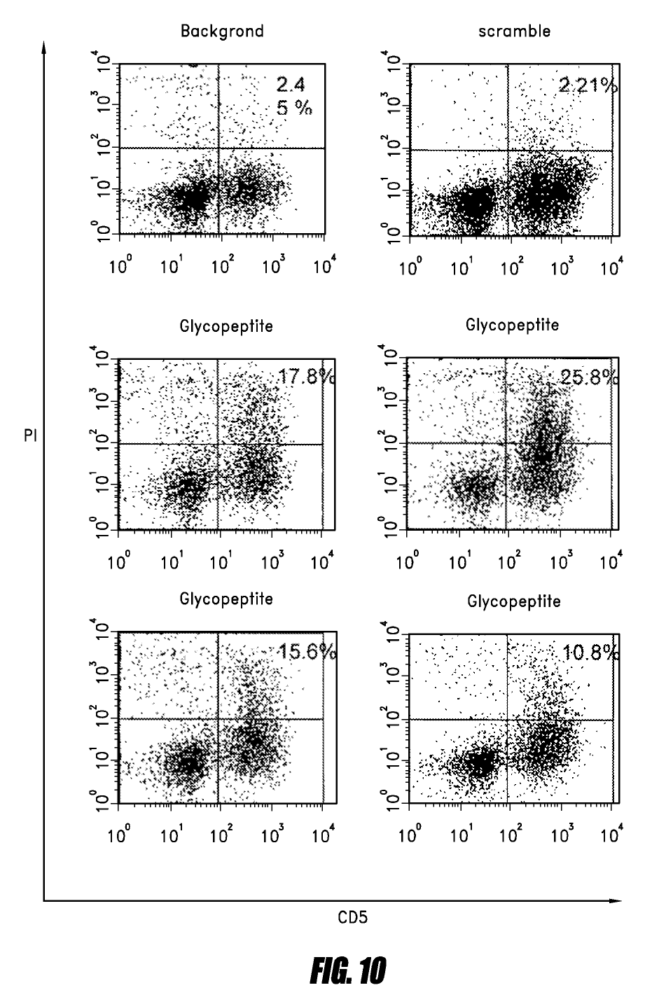

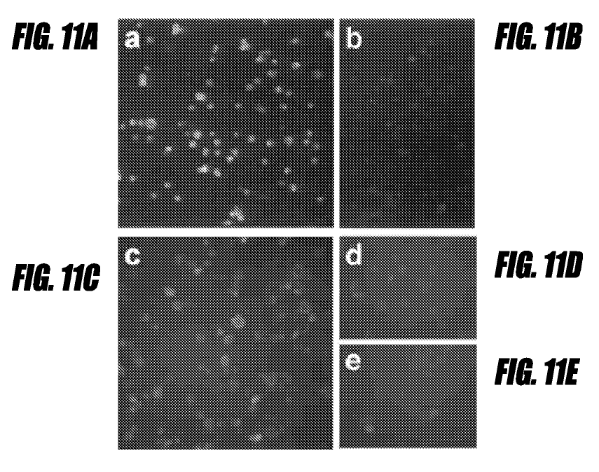

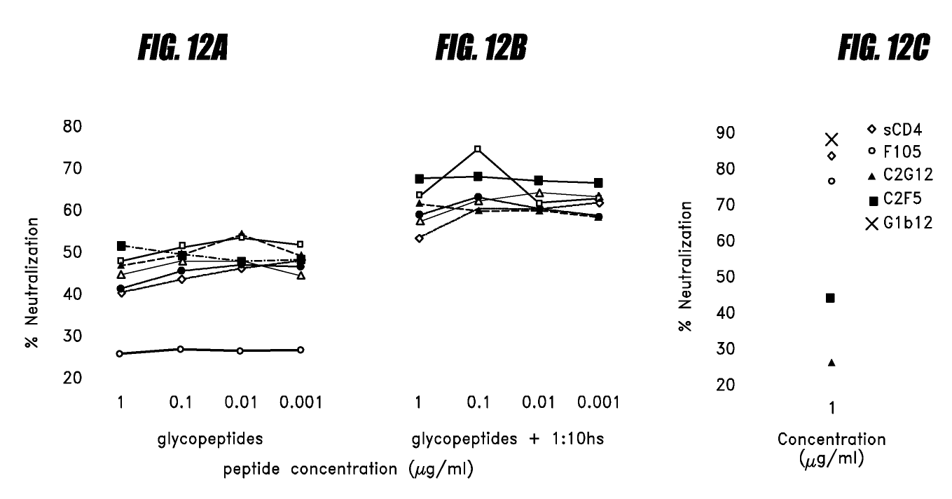

The CD4 glycopeptides were studied for inhibition of cell to cell interactions in a plaque assay system using U87 cells (FIG. 7), in a single round infectivity assay with TZMbl cells (FIG. 7) and in an infectivity assay with H9 cells, wherein multiple rounds of replication were allowed (FIG. 8). Infectivity was performed with the HIV IIIB virus and inactivated human serum from healthy individuals was used as the source of the anti-gal antibody. The data show that CD4 glycopeptides number 3, 4 and 5 (Seq. Id. Nos. 6-8) exhibited a greater neutralization, which may result from the fact that these CD4 glycopeptides overlap a gap of residues that involve a significant amount of interatomic interactions with the gp120 molecule. When using non-heat inactivated human serum in the plaque assay and infectivity assay with H9 cells, it was found that complement contributes to an increase in the neutralization of approximately 30% and 10%, respectively (FIG. 9).

The induction of cellular cytotoxicity facilitated by NK cells was then analyzed on flow cytometry using chronically infected ACH2 cells, that were pre-incubated with the CD4 glycopeptide and 10% of human serum, following by incubation with freshly isolated NK cells at E:T=25:1. Target cells (ACH2 cells) were labeled with an anti-CD5 antibody and toxicity was measured by Propidium Iodide positive cells (FIG. 10). The data show significant induction of an ADCC response in the presence of a CD4 glycopeptide in comparison to background or a scrambled peptide. The next Example describes binding of glycopeptide 4 to the gp120 molecule using immunofluorescence.

### EXAMPLE 6

Binding of glycopeptide 4 (Seq. ID. No. 7) to the gp120 molecule was evaluated by immunofluorescence. Chronically infected ACH2 cells were stimulated with 50 nM of PMA 48 h prior to the performance of the assay to ensure expression of the gp120 molecule on the cell surface. On the day of the assay, 20,000 cells were counted and allowed to dry on a microscope slide and were fixated with a 3% solution of paraformaldehyde at pH 7.0. The slide surface was blocked with a 2% solution human serum albumin (Calbiochem) for 30 min in a humidified chamber at 37° C. Thereafter the glycopeptide was added at a concentration of 5 μg/ml and incubated for 1 h at 37° C. in a humidified chamber. A 1 μg/ml solution of Isolectin B4 Alexa Fluor 488 conjugated (Molecular Probes, Invitrogen) was added to the cells in order to detect the bound peptide and incubated for 1 h at 37° C.

In a second assay that aimed to visualize the glycopeptide-antibody complex, after the incubation of the peptide a 10% dilution of human serum from HIV sero-negative individuals (Cambrex,USA) was added and incubated for 1 h. Then a 2 μg/ml concentration of antihuman IgG and IgM Alexa Fluor conjugated (Molecular Probes, Invitrogen) was added and incubated for 1 h. Cells were checked under a standard fluorescence microscope.

Binding of one of the glycopeptides to surface expressed gp120 was visualized using chronically infected ACH2 cells (FIG. 11). These cells express the gp120 molecule on their surface after stimulation with PMA. Binding of the glycopeptide to the cells was visualized using Alexa Fluor-conjugated isolectin B4, which specifically binds to the gal(α1,3)gal antigen (FIG. 11), and also by using human serum from HIV sero-negative individuals and Alexa Fluor conjugated goat anti-human IgG and IgM FIG. 11C). In the absence of glycopeptide or anti-gal(α1,3)gal antibodies no binding to ACH2 cells was seen (FIGS. 11B and 11D, respectively). The next Example describes a single round neutralization assay using TZM-b1 cells containing a luciferase gene.

### EXAMPLE 7

A single round infectivity neutralization assay was performed using TZM-b1 cells that contain a tat-responsive luciferase reporter gene with the presence of the HIV protease inhibitor Indinavir in the cell culture medium.

Briefly, TZM-b1 10,000 cells/well were pre-seeded in a 96 well plate and incubated overnight at 37° C. The next day the glycopeptide and 150 TCID50 of the IIIB virus were pre-incubated for 1.5 h at 37° C. and thereafter the glycopeptide/HIV mixture was added to the cells together with a 1:10 dilution of inactivated AB human serum obtained from healthy individuals (Cambrex, USA). The cells were incubated with advanced DMEM medium containing 1 mM Indinavir in a final volume of 200 μl/well for 48 h. Each tested concentration of glycopeptide was run in 4 replicates on the same plate. The luciferase activity in individual wells was measured using the Bright Glo Luciferase Kit (Promega, Germany) according to the manufacturer's protocol. The percentage of fusion inhibition was calculated as 1—the ratio of treated wells versus untreated-infected wells multiplied by 100. Monoclonal antibodies G1b12, C2G12, F105 and F425 and soluble CD4 were tested under the same protocol and thus served as positive controls (Trkola A, et al. (1996) Human monoclonal antibody 2G12 defines a distinctive neutralization epitope on the gp120 glycoprotein of human immunodeficiency virus type 1. J Virol 70:1100-1108.; Tuen M, et al. (2005) Characterization of antibodies that inhibit HIV gp120 antigen processing and presentation. Eur J Immunol 35:2541-51, herein expressly incorporated by reference in their entireties).

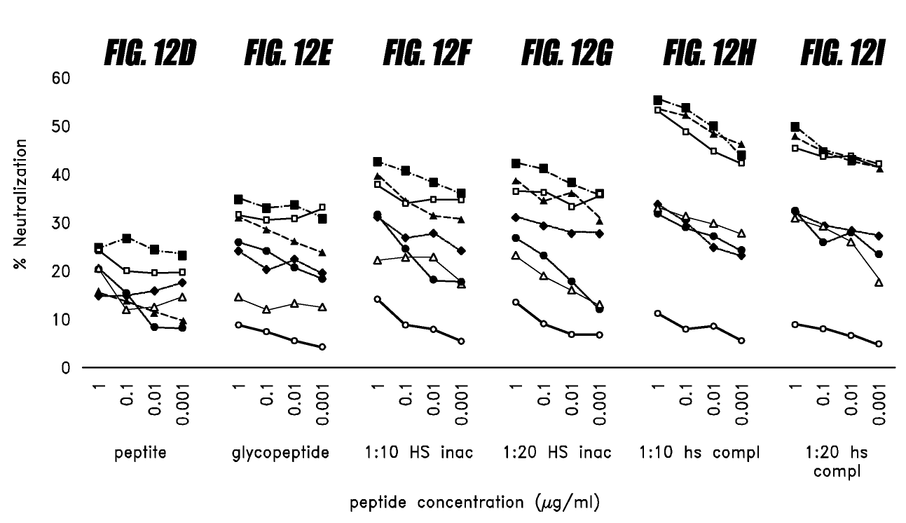

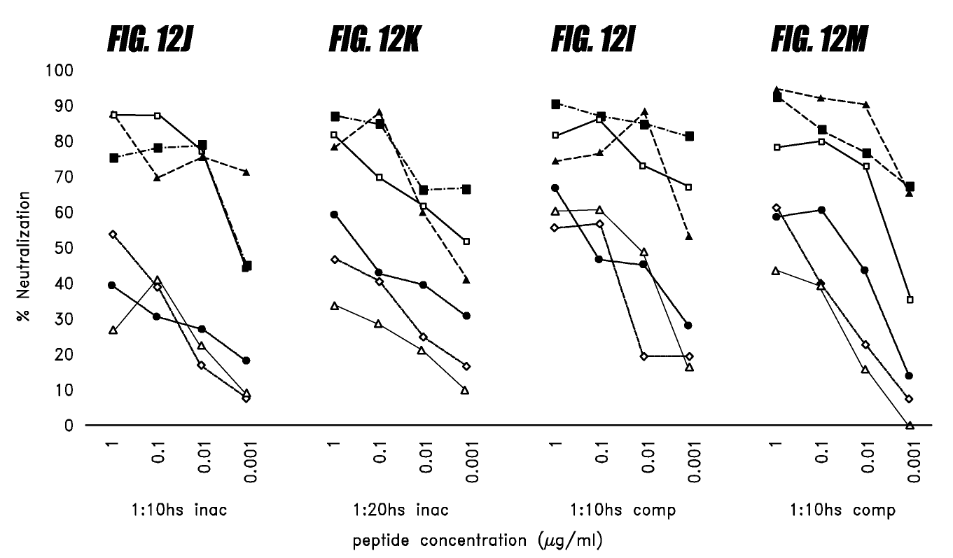

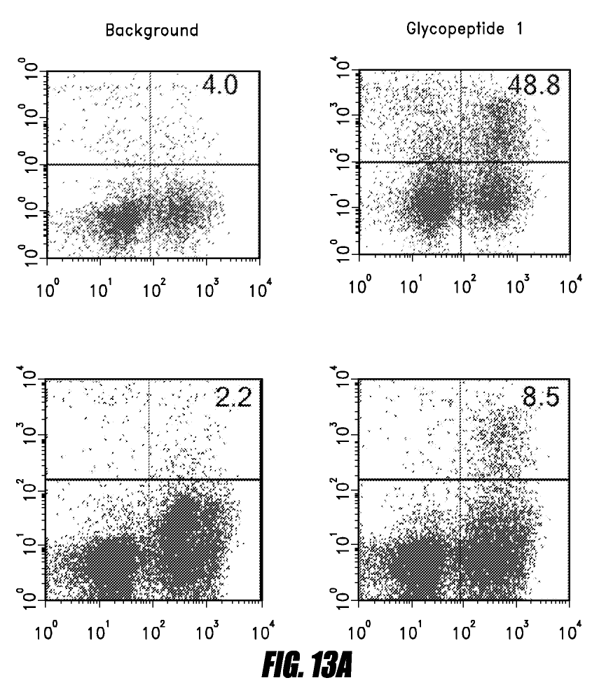

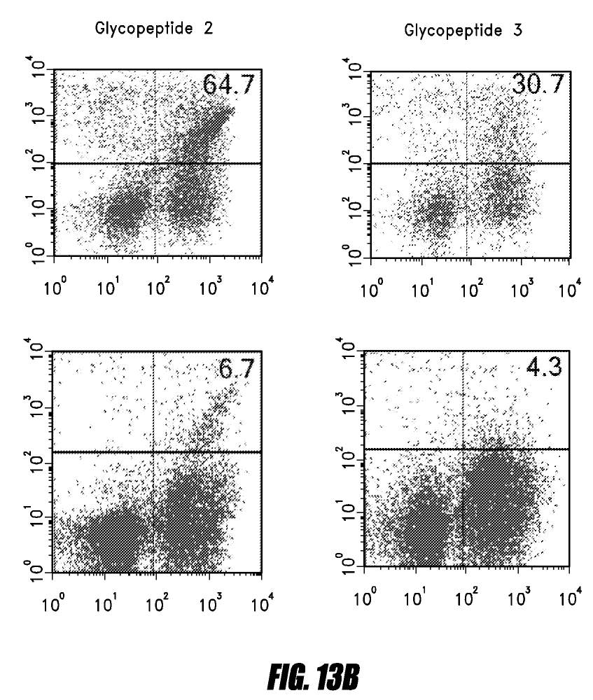

Luminescence values in cell lysate after 48 h of incubation reflect infectivity of the virus added. The glycopeptides were tested alone (FIG. 12A) or in the presence of 10% inactivated human serum from healthy individuals (FIG. 12B). The glycopeptides alone inhibited virus infectivity between 40% and 50%. The addition of human serum increased the neutralization by 10-20 percentage units, giving neutralization values for all the peptides above 50% even at a concentration as low as 1 ng/ml (FIG. 12B). The levels of neutralization seen with the gal(α1,3)gal-linked CD4 peptides were within the same range as those seen with various neutralizing monoclonal antibodies (FIG. 12C). The next Example describes neutralization assays based on the prevention of syncytia formation by HIV-1 infection of adherent U87 glioma cells expressing the CD4 receptor and the CXCR4 co-receptor.

### EXAMPLE 8

Neutralization was tested based on the prevention of syncytia formation by HIV-1 infection of adherent U87 glioma cells expressing the CD4 receptor and the CXCR4 co-receptor, previously described by Shi Y, et al., (2002) A new cell line-based neutralization assay for primary HIV type 1 isolates. AIDS Res Hum Retroviruses 18:957-967, herein expressly incorporated by reference in its entirety.

Cells were seeded into 48 well plates (Costar, Corning USA) at a concentration of 10⁵ cell/well one day prior to infection. On the day of infection, glycopeptides, pooled AB type human serum from healthy individuals (Cambrex, USA) and previously titrated HIV IIIB virus were diluted in culture medium and pre-incubated in a separate 48 well plate for 1 h at 37° C. The peptides were analyzed at different concentrations to evaluate the minimum dose required for antiviral activity. Two hundred microliters of each dilution were distributed into triplicate wells containing the U87 CD4⁺-CXCR4⁺ cells and incubated for 12 h after which medium was removed and 1 ml of D-MEM medium was added to the cells. The positive controls were infected cells in the presence or absence of human serum and negative controls consisted of uninfected cells in the presence or absence of human serum and an irrelevant glycosylated peptide. At day five the cells were fixated with methanol-acetone (1:1). The number of plaque-forming units was determined after haematoxylin staining and the index was extracted from the ratio between infected cells treated with the peptides and human serum and cells infected with the virus in the presence of human serum. Each peptide was tested at four different concentrations, in quadruplicates in the same plate and in five different plates, i.e. each peptide was run in twenty wells at each concentration.

The fusion of infected cells is mediated by the gp120/gp41 proteins of HIV-1 expressed on the surface of infected cells that bind to CD4 and CXCR4 on neighboring cells. The addition of non-glycosylated or gal(α1,3)gal-linked CD4 peptides resulted in <40% neutralization, suggesting that the peptides themselves are inefficient in preventing syncytia formation (FIGS. 12D and 12E). When heat inactivated human serum was added the level of inhibition increased 5-10 percentage units, suggesting that the presence of natural antibodies and gal(α1,3)gal-linked CD4 peptides alone have a limited effect on the inhibition of syncytia formation (FIGS. 12F and 12G). However, when non-inactivated human sera was added together with the gal(α1,3)gal-linked CD4 peptides the levels of inhibition of syncytia formation increased for all the glycosylated-CD4 peptides, but not for the gal(α1,3)gal-linked control peptide (FIG. 12). Overall, the efficiency of the neutralizing activity was improved by 50-100% for all peptides when both the anti-gal(α1,3)gal antibodies and complement was present, strongly suggesting that the biological activity of the natural antibody had been redirected to HIV-1. Highest neutralization values were obtained with peptides 3, 4 and 5 (FIGS. 12H and 12I). The next Example describes an infectivity assay using H9 cells infected with HIV IIIB virus.

### EXAMPLE 9

An infectivity assay was performed on H9 cells that were infected with HIV IIIB virus. All peptides were run at four different dilutions and each dilution was run in quadruplicates together with a 10% or 5% of pooled AB human serum from healthy individuals. (Cambrex, USA).

H9 cells were infected with 100 TCID₅₀ of the HIV IIIB virus and incubated for 1.5 h at 37° C. after which they were centrifuged, the supernatant was discarded and the cells were re-suspended in RPMI medium for a final concentration of 200,000 cells/ml.

One 48 well plate was run per glycopeptide, containing the four dilutions of the glycopeptide and the four replicates of each one of them, plus a positive control of infected cells (with and without human serum) and a negative control of non-infected H9 cells. Each well contained 500 μl of the infected cells +500 μl of the diluted peptide and the chosen dilution of the human serum.

Plates were incubated at 37° C.+5% CO₂ until day 7 when 500 μl of supernatant were collected from each well and stored at −20 degrees until further analysis. An additional 500 μl of fresh RPMI medium with the appropriate dilution of the peptide plus the human serum were added to the wells and incubated at 37 degrees until day 11 when the supernatant was measured for Reverse Transcriptase (RT) activity (Cavidi, Sweden) according to the manufacturer's protocol.

All peptides neutralized the infection in a concentration dependent manner, but again peptides 3, 4, and 5 displayed the highest values giving a 90% neutralization at 1 μg/ml of glycopeptides and more than 50% decrease in the infectivity at a 0.01 μg/ml glycopeptide concentration (corresponding to approximately 5 nM) at a 5 or 10% concentration of human serum (FIGS. 12J and 12K). In this assay, the presence of active complement gave an increase of neutralization by approximately 10% as compared to heat-inactivated human serum (FIGS. 12L and 12M). Thus, in this assay when multiple rounds of the whole viral life cycle were allowed, the effect of complement was less pronounced. The next Example describes the induction of ADCC response to HIV infected cells bound to glycopeptides.

### EXAMPLE 10

The Fc region of antibodies attached to the surface of HIV-1 infected cells can be recognized by the CD16 receptor of NK cells and consecutively trigger a cytolytic response that will ultimately lead to the apoptosis of the targeted cell. To test whether anti-gal(α1,3)gal antibodies could induce ADCC of HIV infected cells when bound to the glycopeptides, flow cytometry was conducted on stimulated ACH2 cells expressing gp120. ACH2 cells were stimulated with 50 mM PMA (Sigma) 48 h prior to the assay in order to induce virus production. On the day of the assay the ACH2 cells were pre-incubated for 2 h with the diluted glycopeptide and 10% human serum AB from healthy individuals (Cambrex, USA). Freshly isolated NK cells were added at a final effector:target (E:T) ratio of 25:1. During the final 15 min of incubation the ACH2 cells were stained by adding 5 μL/sample of anti CD5-APC conjugated antibody (BD biosciences) and as a marker of cell death, propidium iodide (Invitrogen) was added at 1 μg/ml. Samples were analyzed using the Cell Quest Program and live ACH2 cells were gated by size.

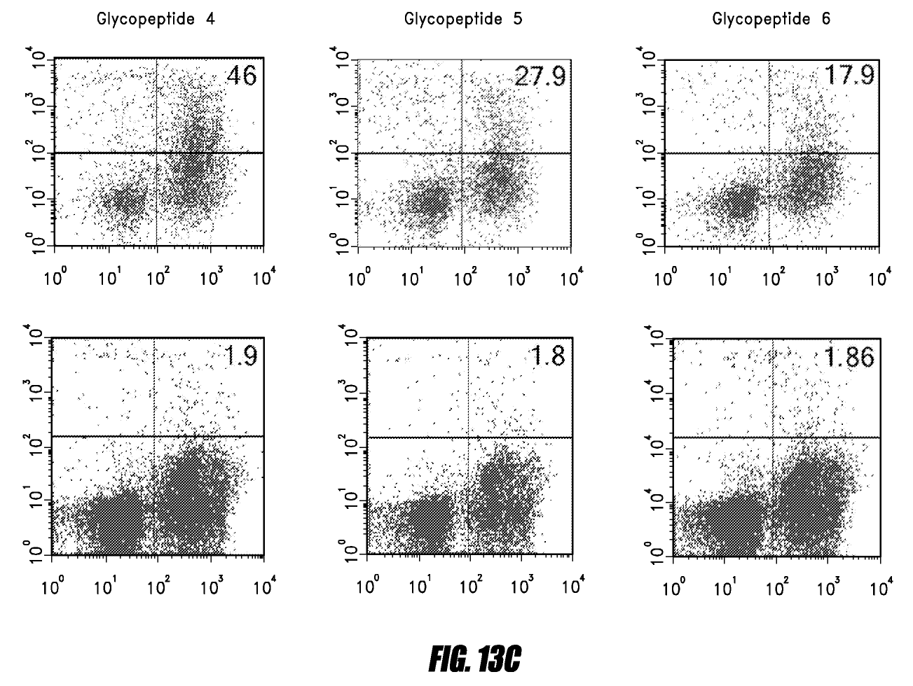

NK cell-mediated killing of HIV-1 infected cells was dependent on the concentration of the glycopeptides (data not shown) and lower concentrations (<10 μg/mL) did not display a significant portion of double positive cells as compared to the background. NK cell-dependent cytolysis was obtained with all gal(α1,3)gal-linked CD4 peptides but only in the presence of human serum (FIGS. 13A-C top row) strongly suggesting that also the NK cell-binding activity of the natural antibodies had been redirected to HIV-1 infected cells (FIG. 13). In conclusion, the gal α (1,3) gal β-linked CD4 peptides were clearly able to redirect the ADCC activity mediated by the anti-gal α (1,3) gal β antibodies to target HIV-1 infected lymphocytes.

The section below describes several pharmaceuticals and medicaments comprising specificity exchangers that comprise saccharides and/or glycoconjugates.

Pharmaceuticals Comprising Specificity Exchangers that Comprise Saccharides and/or Glycoconjugates

The specificity exchangers described herein are suitable for incorporation into pharmaceuticals for administration to subjects in need of a compound that treats or prevents infection by a pathogen. These pharmacologically active compounds can be processed in accordance with conventional methods of galenic pharmacy to produce medicinal agents for administration to mammals including humans. The active ingredients can be incorporated into a pharmaceutical product with and without modification. Further, the manufacture of pharmaceuticals or therapeutic agents that deliver the pharmacologically active compounds of this invention by several routes are aspects of the present invention. For example, and not by way of limitation, DNA, RNA, and viral vectors having sequences encoding a specificity exchanger described herein are used with embodiments of the invention. Nucleic acids encoding the embodied specificity exchangers can be administered alone or in combination with other active ingredients.

The specificity exchangers can be employed in admixture with conventional excipients, i.e., pharmaceutically acceptable organic or inorganic carrier substances suitable for parenteral, enteral (e.g., oral) or topical application that do not deleteriously react with the pharmacologically active ingredients described herein. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more vehicles that can be used are described in Remmington's Pharmaceutical Sciences, 15th Edition, Easton:Mack Publishing Company, pages 1405-1412 and 1461-1487 (1975) and The National Formulary XIV, 14th Edition, Washington, American Pharmaceutical Association (1975), herein incorporated by reference. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like so long as the auxiliary agents does not deleteriously react with the specificity exchangers.

The effective dose and method of administration of a particular pharmaceutical having a specificity exchanger that comprises a plurality of saccharides and/or glycoconjugates can vary based on the individual needs of the patient and the treatment or preventative measure sought. Therapeutic efficacy and toxicity of such compounds can be determined by standard pharmaceutical procedures in cell cultures or experimental animals, e.g., ED50 (the dose therapeutically effective in 50% of the population). For example, the effective dose of a specificity exchanger can be evaluated using the characterization assays described above. The data obtained from these assays is then used in formulating a range of dosage for use with other organisms, including humans. The dosage of such compounds lies preferably within a range of circulating concentrations that include the ED50 with no toxicity. The dosage varies within this range depending upon type of specificity exchanger, the dosage form employed, sensitivity of the organism, and the route of administration.

Normal dosage amounts of a specificity exchanger can vary from approximately 1 to 100,000 micrograms, up to a total dose of about 10 grams, depending upon the route of administration. Desirable dosages include about 250 mg-1 mg, about 50 mg-200 mg, and about 250 mg-500 mg.

In some embodiments, the dose of a specificity exchanger preferably produces a tissue or blood concentration or both from approximately 0.1 μM to 500 μM. Desirable doses produce a tissue or blood concentration or both of about 1 to 800 μM. Preferable doses produce a tissue or blood concentration of greater than about 10 μM to about 500 μM. Although doses that produce a tissue concentration of greater than 800 μM are not preferred, they can be used. A constant infusion of a specificity exchanger can also be provided so as to maintain a stable concentration in the tissues as measured by blood levels.

The exact dosage is chosen by the individual physician in view of the patient to be treated. Dosage and administration are adjusted to provide sufficient levels of the active moiety or to maintain the desired effect. Additional factors that can be taken into account include the severity of the disease, age of the organism being treated, and weight or size of the organism, diet, time and frequency of administration, drug combination(s), reaction sensitivities, and tolerance/response to therapy. Short acting pharmaceutical compositions are administered daily or more frequently whereas long acting pharmaceutical compositions are administered every 2 or more days, once a week, or once every two weeks or even less frequently.

Routes of administration of the pharmaceuticals include, but are not limited to, topical, transdermal, parenteral, gastrointestinal, transbronchial, and transalveolar. Transdermal administration is accomplished by application of a cream, rinse, gel, etc. capable of allowing the specificity exchangers to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions having the specificity exchangers described herein that are suitable for transdermal or topical administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al., herein expressly incorporated by reference in its entirety.

Compositions having the specificity exchangers described herein that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the specificity exchangers described herein that are suitable for transbronchial and transalveolar administration include, but are not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the specificity exchangers described herein.

Compositions having the specificity exchangers described herein that are suitable for gastrointestinal administration include, but not limited to, pharmaceutically acceptable powders, pills or liquids for ingestion and suppositories for rectal administration. Due to the ease of use, gastrointestinal administration, particularly oral, is a preferred embodiment. Once the pharmaceutical comprising the specificity exchanger has been obtained, it can be administered to an organism in need to treat or prevent pathogenic infection.

Aspects of the invention also include a coating for medical equipment such as prosthetics, implants, and instruments. Coatings suitable for use on medical devices can be provided by a gel or powder containing the specificity exchanger or by a polymeric coating into which a specificity exchanger is suspended. Suitable polymeric materials for coatings of devices are those that are physiologically acceptable and through which a therapeutically effective amount of the specificity exchanger can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, herein expressly incorporated by reference in its entirety. The section below describes methods of treating and preventing disease using the specificity exchangers described herein.

Treatment and Prevention of Disease Using a Specificity Exchanger that Comprises a Plurality of Saccharides and/or a Glycoconjugate

Pharmaceuticals comprising the specificity exchangers described herein can be administered to a subject in need to treat and/or prevent infection by a pathogen that has a receptor. Such subjects in need can include individuals at risk of contacting a pathogen or individuals who are already infected by a pathogen. These individuals can be identified by standard clinical or diagnostic techniques.

By one approach, a subject in need of an agent that inhibits viral infection can be administered a specificity exchanger that recognizes a receptor present on the particular etiologic agent. Accordingly, a subject in need of an agent that inhibits viral infection is identified by standard clinical or diagnostic procedures. Next, the subject in need is provided a therapeutically effective amount of a specificity exchanger that interacts with a receptor present on the type of virus infecting the individual. As above, it may be desired to determine whether the subject has a sufficient titer of antibody to interact with the antigenic domain of the specificity exchanger prior to administering the specificity exchanger.

For example, a subject infected with HIV-1 is identified as a subject in need of an agent that inhibits proliferation of a pathogen. This subject is then provided a therapeutically effective amount of a specificity exchanger described herein. The specificity exchanger used in this method comprises a specificity domain that interacts with a receptor present on the virus (e.g., gp120). In some embodiments, the specificity exchanger also comprises an antigenic domain that has a plurality of saccharides and/or glycoconjugates, which are recognized by high titer antibodies present in the subject in need. It may also be desired to screen the subject in need for the presence of high titer antibodies that recognize the antigenic domain prior to providing the subject the specificity exchanger. This screening can be accomplished by EIA or ELISA using immobilized antigenic domain or specificity exchanger, as described above.

For example, a subject infected with HIV-1 having a CD4 cell count high enough to undergo therapy comprising T cell destruction through ADCC is identified as a subject in need of an agent that induces ADCC of HIV-1 infected T-cells. This subject is then provided a therapeutically effective amount of a specificity exchanger described herein. The specificity exchanger used in this method comprises a specificity domain that interacts with a receptor present on the virus (e.g., gp120). In some embodiments, the specificity exchanger also comprises an antigenic domain that has a plurality of saccharides and/or glycoconjugates, which are recognized by high titer antibodies present in the subject in need. It may also be desired to screen the subject in need for the presence of high titer antibodies that recognize the antigenic domain prior to providing the subject the specificity exchanger. This screening can be accomplished by EIA or ELISA using immobilized antigenic domain or specificity exchanger, as described above.

In the same vein, a subject in need of an agent that inhibits the proliferation of cancer can be administered a specificity exchanger that interacts with a receptor present on the cancer cell. For example, a subject in need of an agent that inhibits proliferation of cancer is identified by standard clinical or diagnostic procedures; then the subject in need is provided a therapeutically effective amount of a specificity exchanger that interacts with a receptor present on the cancer cells infecting the subject. As noted above, it may be desired to determine whether the subject has a sufficient titer of antibody to interact with the antigenic domain of the specificity exchanger prior to administering the specificity exchanger.

The specificity exchangers described herein can also be administered to subjects as a prophylactic to prevent the onset of disease. Virtually anyone can be administered a specificity exchanger described herein for prophylactic purposes, (e.g., to prevent a bacterial infection, viral infection, or cancer). It is desired, however, that subjects at a high risk of contracting a particular disease are identified and provided a specificity exchanger. Subjects at high risk of contracting a disease include individuals with a family history of disease, the elderly or the young, or individuals that come in frequent contact with a pathogen (e.g., health care practitioners). Accordingly, subjects at risk of becoming infected by a pathogen that has a receptor are identified and then are provided a prophylactically effective amount of specificity exchanger.

One prophylactic application for the specificity exchangers described herein concerns coating or cross-linking the specificity exchanger to a medical device or implant. Implantable medical devices tend to serve as foci for infection by a number of bacterial species. Such device-associated infections are promoted by the tendency of these organisms to adhere to and colonize the surface of the device. Consequently, there is a considerable need to develop surfaces that are less prone to promote the adverse biological reactions that typically accompany the implantation of a medical device.

By one approach, the medical device is coated in a solution of containing a specificity exchanger. Prior to implantation, medical devices (e.g., a prosthetic valve) can be stored in a solution of specificity exchangers, for example. Medical devices can also be coated in a powder or gel having a specificity exchanger. For example, gloves, condoms, and intrauterine devices can be coated in a powder or gel that contains a specificity exchanger that interacts with a bacterial or viral receptor. Once implanted in the body, these specificity exchangers provide a prophylactic barrier to infection by a pathogen.

In some embodiments, the specificity exchanger is immobilized to the medical device. As described above, the medical device is a support to which a specificity exchanger can be attached. Immobilization may occur by hydrophobic interaction between the specificity exchanger and the medical device but a preferable way to immobilize a specificity exchanger to a medical device involves covalent attachment. For example, medical devices can be manufactured with a reactive group that interacts with a reactive group present on the specificity exchanger.

By one approach, a periodate is combined with a specificity exchanger comprising a 2-aminoalcohol moiety to form an aldehyde-functional exchanger in an aqueous solution having a pH between about 4 and about 9 and a temperature between about 0 and about 50 degrees Celsius. Next, the aldehyde-functional exchanger is combined with the biomaterial surface of a medical device that comprises a primary amine moiety to immobilize the specificity exchanger on the support surface through an imine moiety. Then, the imine moiety is reacted with a reducing agent to form an immobilized specificity exchanger on the biomaterial surface through a secondary amine linkage. A similar chemistry can be used to attach the sugar to the support and/or specificity domain of the specificity exchanger, as well. Other approaches for cross-linking molecules to medical devices, (such as described in U.S. Pat. No. 6,017,741, herein expressly incorporated by reference in its entirety), can be modified to immobilize the specificity exchangers described herein.

Although the invention has been described with reference to embodiments and examples, it should be understood that various modifications can be made without departing from the spirit of the invention. Accordingly, the invention is limited only by the following claims. All references cited herein are hereby expressly incorporated by reference in their entireties.

What is claimed is:

1. A method of inducing an antibody dependent cellular cytotoxicity (ADCC) in a subject in need thereof, comprising:
identifying a subject in need of an ADCC response against HIV infected cells, wherein said subject has a CD4 cell count that allow safe induction of an ADCC response against CD4 infected cells;
providing to said identified subject an effective amount of a glycoconjugate peptide comprising a binding fragment of a CD4 receptor for HIV gp120 synthetically conjugated to gal α (1,3) gal P; and
measuring the reduction of HIV viral load in said subject.

2. The method of claim 1, wherein said subject is evaluated for presence of natural antibodies specific for gal α (1,3) gal β before administration of said glycoconjugate peptide.

3. The method of claim 1, wherein said gal α (1,3) gal β is synthetically conjugated to said binding fragment of a CD4 receptor for HIV gp120 by attachment at one amino acid.

4. The method of claim 1, wherein said binding fragment of a CD4 receptor for HIV gp120 is less than 200 amino acids in length.

5. The method of claim 1, wherein said binding fragment of a CD4 receptor for HIV gp120 is less than 150 amino acids in length.

6. The method of claim 1, wherein said binding fragment of a CD4 receptor for HIV gp120 is less than 100 amino acids in length.

7. The method of claim 1, wherein said binding fragment of a CD4 receptor for HIV gp120 is less than 50 amino acids in length.

8. The method of claim 1, wherein said binding fragment of a CD4 receptor for HIV gp120 is less than 25 amino acids in length.

9. The method of claim 1, wherein said binding fragment of a CD4 receptor for HIV gp120 is less than or equal to 15 amino acids in length.

10. The method of claim 1, wherein said gal α (1,3) gal β is synthetically conjugated to a hydroxylated amino acid.

11. The method of claim 1, wherein said gal α (1,3) gal β is synthetically conjugated by an NH₂-linkage.

12. The method of claim 1, wherein said gal α (1,3) gal β is synthetically conjugated to the N-terminal end of said binding fragment of a CD4 receptor for HIV gp120.

13. The method of claim 3, wherein said gal α (1,3) gal β is synthetically conjugated to a hydroxylated amino acid.

14. The method of claim 3, wherein said gal α (1,3) gal β is synthetically conjugated by an NH₂-linkage.

15. The method of claim 3, wherein said gal α (1,3) gal β is synthetically conjugated to the N-terminal end of said binding fragment of a CD4 receptor for HIV gp120.

16. The method of claim 1, wherein said subject is a human.

17. The method of claim 1, further comprising measuring ADCC of said infected cells mediated by NK cells.

18. A method of neutralizing Human Immunodeficiency Virus (HIV) infection comprising:
identifying an HIV infected cell; and
contacting said cell with a glycoconjugate peptide comprising a binding fragment of a CD4 receptor for HIV gp120 synthetically conjugated to gal α (1,3) gal P; and,
measuring neutralization of HIV.

19. The method of claim 18, wherein said gal α (1,3) gal p is synthetically conjugated to said binding fragment of a CD4 receptor for HIV gp120 by attachment at one amino acid.

20. The method of claim 18, wherein said binding fragment of a CD4 receptor for HIV gp120 is less than 200 amino acids in length.

21. The method of claim 18, wherein said binding fragment of a CD4 receptor for HIV gp120 is less than 150 amino acids in length.

22. The method of claim 18, wherein said binding fragment of a CD4 receptor for HIV gp120 is less than 100 amino acids in length.

23. The method of claim 18, wherein said binding fragment of a CD4 receptor for HIV gp120 is less than 50 amino acids in length.

24. The method of claim 18, wherein said binding fragment of a CD4 receptor for HIV gp120 is less than 25 amino acids in length.

25. The method of claim 18, wherein said binding fragment of a CD4 receptor for HIV gp120 is less than or equal to 15 amino acids in length.

26. The method of claim 18, wherein said gal α (1,3) gal 13 is synthetically conjugated to a hydroxylated amino acid.

27. The method of claim 18, wherein said gal α (1,3) gal β is synthetically conjugated by an NH₂-linkage.

28. The method of claim 18, wherein said gal α (1,3) gal β is synthetically conjugated to the N-terminal end of said binding fragment of a CD4 receptor for HIV gp120.

29. The method of claim 19, wherein said gal α (1,3) gal β is synthetically conjugated to a hydroxylated amino acid.

30. The method of claim 19, wherein said gal α (1,3) gal β is synthetically conjugated by an NH₂-linkage.

31. The method of claim 19, wherein said gal α (1,3) gal β is synthetically conjugated to the N-terminal end of said binding fragment of a CD4 receptor for HIV gp120.

32. A method of inducing an antibody dependent cellular cytotoxicity (ADCC) response against an HIV infected cell comprising:
indentifying an HIV infected cell;
contacting said cell with an effective amount of a glycoconjugate peptide comprising a binding fragment of a CD4 receptor for HIV gp120 synthetically conjugated to gal α (1,3) gal β; and
measuring an ADCC response against said HIV infected cell.

33. The method of claim 32, wherein said measuring of the ADCC response comprises a measurement of cellular cytotoxicity mediated by NK cells.

34. The method of claim 32, wherein said gal α (1,3) gal β is synthetically conjugated to said binding fragment of a CD4 receptor for HIV gp120 by attachment at one amino acid.

35. The method of claim 32, wherein said binding fragment of a CD4 receptor for HIV gp120 is less than 200 amino acids in length.

36. The method of claim 32, wherein said binding fragment of a CD4 receptor for HIV gp120 is less than 150 amino acids in length.

37. The method of claim 32, wherein said binding fragment of a CD4 receptor for HIV gp120 is less than 100 amino acids in length.

38. The method of claim 32, wherein said binding fragment of a CD4 receptor for HIV gp120 is less than 50 amino acids in length.

39. The method of claim 32, wherein said binding fragment of a CD4 receptor for HIV gp120 is less than 25 amino acids in length.

40. The method of claim 32, wherein said binding fragment of a CD4 receptor for HIV gp120 is less than or equal to 15 amino acids in length.

41. The method of claim 32, wherein said gal α (1,3) gal β is synthetically conjugated to a hydroxylated amino acid.

42. The method of claim 32, wherein said gal α (1,3) gal β is synthetically conjugated by an NH₂-linkage.

43. The method of claim 32, wherein said gal α (1,3) gal β is synthetically conjugated to the N-terminal end of said binding fragment of a CD4 receptor for HIV gp120.

44. The method of claim 34, wherein said gal α (1,3) gal β is synthetically conjugated to a hydroxylated amino acid.

45. The method of claim 34, wherein said gal α (1,3) gal β is synthetically conjugated by an NH₂-linkage.

46. The method of claim 34, wherein said gal α (1,3) gal β is synthetically conjugated to the N-terminal end of said binding fragment of a CD4 receptor for HIV gp120.

47. The method of claim 32, wherein said HIV infected cell is present in a human.
